(19) United States
(12) Patent Application Publication
(10) Pub. No.: US 2025/0281044 A1
(43) Pub. Date: Sep. 11, 2025

(54) On-Demand Functionalized Textiles for Drag-and-Drop, Near Field Multi-Body Area Networks

(71) Applicant: The Regents of the University of California, Oakland, CA (US)

(72) Inventors: Peter Tseng, Irvine, CA (US); Amirhossein Hajiaghajani, Irvine, CA (US)

(21) Appl. No.: 18/289,565

(22) PCT Filed: May 11, 2022
(86) PCT No.: PCT/US22/28858

§ 371 Date: Nov. 5, 2023

Related U.S. Application Data
(60) Provisional application No. 63/187,668, filed on May 12, 2021.

(51) Int. Cl. A61B 5/00; A61B 5/11; H04B 13/00; H04W 84/10

(52) U.S. Cl. CPC A61B 5/0024; A61B 5/1123; A61B 5/6804; H04B 13/005; A61B 2560/0462; H04W 84/10

(57) ABSTRACT

On-demand functionalized textiles for near field body area networks in accordance with embodiments of the invention are disclosed. In one embodiment, a body area network is provided, the body area network including: a first array of magnetically coupled resonators configured to propagate magneto-inductive (MI) surface waves, where the first array of magnetically coupled resonators comprises a plurality of MI elements, and where the first array of magnetically coupled resonators creates a flexible magnetic metamaterial path for wireless communication using the MI surface waves.

### CROSS-REFERENCE TO RELATED APPLICATION

The current application is a National Stage of International Application No PCT/US22/28858, which claims priority to U.S. Provisional Patent Application No. 63/187,668 filed on May 12, 2021, the disclosures of which are incorporated herein by reference.

### FEDERAL FUNDING SUPPORT

This invention was made with Government support under Grant No. ECCS-1942364, awarded by the National Science Foundation. The Government has certain rights in the invention.

### FIELD OF THE INVENTION

The present invention generally relates to wireless communications and more specifically to on-demand functionalized textiles (may also be referred to as “textile-integrated metamaterials”) for near field body area networks.

### BACKGROUND

Health monitoring and activity-tracking technologies rely on wearable or implantable sensors that link to different regions of the human body and/or link multiple bodies. These sensors may be used to create multi-node networks that interpret information from our bodies and objects that may be interacted with. In order to parse biometric information in real time, such networks should include secure and reliable communication links between nodes.

### SUMMARY OF THE INVENTION

The various embodiments of the present on-demand functionalized textiles for near field multi-body area networks (may also be referred to as “near field BAN” or “BAN” or “body area networks”) contain several features, no single one of which is solely responsible for their desirable attributes. Without limiting the scope of the present embodiments, their more prominent features will now be discussed below. In particular, the present functionalized textiles for near field multi-body area networks will be discussed in the context of textiles (e.g., shirts and pants). However, the use of textiles is merely exemplary and various other materials and/or particular textiles may be utilized for near field BANs as appropriate to the requirements of a specific application in accordance with various embodiments of the invention. Further, the present functionalized textiles for near field BANs will be discussed in the context of particular sensors (e.g., NFC sensors, biosensors, etc.). However, use of particular sensors are also merely exemplary and various other sensors utilizing particular communication protocols and/or for detecting and/or measuring a variety of physical properties may be utilized for functionalized textiles for near field BANs as appropriate to the requirements of a specific application in accordance with various embodiments of the invention. After considering this discussion, and particularly after reading the section entitled “Detailed Description,” one will understand how the features of the present embodiments provide the advantages described here.

One aspect of the present embodiments includes the realization that the robustness of a BAN may be dependent on a number of characteristics, including the level of comfort (or the size/weight of the device nodes), adaptability to pre-existing clothing, compliance with commonly-used standards, node sampling rates, and wireless powering capabilities. Further, robustness of a BAN may also be dependent on the ability to communicate with nearby BANs and local area networks, creating seamless connections with peripheral internet-of-things (IoT) without compromising security. Typically, a BAN may offer such communication capabilities while being light-weight and battery-free (to minimize user burden and facilitate truly continuous monitoring) while being able to sample dynamically-placed wireless nodes at high rates relevant to our unpredictable daily lives. BANs have traditionally been equipped with over-the-air communications such as custom radio frequency (RF) transducers, RF identification, or Bluetooth. However, such radiative approaches often suffer from high power consumption and relatively low levels of security. This is because despite coding methods, a nearby third-party receiver may listen to the communication between the reader or sensing nodes. This issue may be addressed by limiting the operational range of such communication links to ensure the target receiver/transmitters are placed spatially close enough to the body. For example, one solution to this challenge is to convert/replace the far field radiation unit (such as the planar inverted F-antenna in Bluetooth technology) with a near field antenna and employ surface waves (instead of spatial radiation). This, however, may require exclusively developed Bluetooth sensors as well as a modified reader (often mobile phone) with irregular antennas, which limits wider application. In addition, Bluetooth power transfer may be limited and batteries are typically required at sensing nodes to accurately sample the environment.

Another aspect of the present embodiments includes the realization that based on Bluetooth technology, surface-plasmon-like metamaterial networks made out of laser-cut conductive fabrics and attached on clothing with textile adhesive may enable surface-bound magnetic wave propagation at 2.4 GHz. However, such networks must exhibit continuous conductivity across their entire length scale (otherwise significant transmission loss occurs) and require relatively complex sewing steps onto clothing. The electrical characteristics of these BANs at microwave frequencies also exhibit rather large sensitivity to the presence of tissues. Such BAN architectures with majorly continuous conductivity across the array cannot be readily reconfigured onto pre-existing clothing and suffer from difficult clothing attachment methods, thus limiting their practical utility and making network expansion difficult (user may need wireless sensing nodes in new body areas). Furthermore, the embroidered spoof surface plasmon (SPP) metamaterials may not be suitable for below GHz as they may require larger dimensions at lower frequencies (at NFC). Moreover, the enhanced security enabled by redirecting transmitted energy to guided modes (instead of conventional long-range far field radiation) does not allow third-party devices to listen to the Bluetooth communication. This redirection is not often fully efficient, resulting in power leakage at microwave frequencies and ultimately challenging the signal transmission. Although custom Bluetooth antennas with nearfield emission profile may be available, they are not currently used in commercial Bluetooth sensors and/or readers and may violate relevant regulation standards. Finally, in such spoof surface plasmon structures the wave propagates on and very close to the surface, and limits propagation from object to object that could occur between pieces of clothing or nearby BANs.

Another aspect of the present embodiments includes the realization that another approach to enhance the BAN security, which does not require alteration from relevant standards, may be to use near field emitting devices such as near field communication (NFC) or Qi-based technologies. For example, a single reader may utilize several near field emitting hot spots (that are connected by wire) to transmit signals to often battery-free wireless nodes placed around the body. An advantage of NFC over the Bluetooth protocol may be its plug-and-play ability to pair communication nodes to the reader seamlessly, in addition to supporting wireless power transfer. This enables pick-and-place characteristics that allow wireless sensing nodes to be switched at will—such capability reduces user burden and has significant convenience in day-to-day use.

Another aspect of the present embodiments includes the realization that with the near field regime, research has focused on connecting a few loop antennas (known as hubs and terminals) using embroidered conductive thread or metal wires. In such structures, the NFC transponders may pair only if placed close to these static hot spots. The wire-linked nature of this approach also makes it unsuitable for on-demand expansion due to long wires used to connect the disparate terminals. Although wireless wearable inter-coil communication has been shown to connect two wristband-like coils, it suffers from a short operational range. Similarly, the low-frequency capacitive signaling of human skin and muscle can impede powering of battery-free sensor nodes and may be easily disrupted by or interfere with immediate surroundings (particularly conductive substances).

Another aspect of the present embodiments includes the realization that electromagnetic surface wave propagation (often achieved via metamaterials) operating at low frequency bands could potentially be used to create an extendable and wireless BAN at NFC bands. Electromagnetic metamaterials typically function at relatively large frequencies (above GHz) and may be fabricated in rigid forms to ensure their perfectly periodic structure. Magneto-inductive (also known as magnetic metamaterials) structures have, in particular, been shown to allow wave propagation, enabling a communication path bounded to an array of magnetically coupled resonators (conventionally an array of split rings). Their utility may range from high frequency metasurfaces applications to wireless power transfer. Due to the magnetically coupled nature of such resonators, and unlike spoof surface plasmon structures, magneto-inductive arrays may be wirelessly connected. Magneto-inductive structures with three dimensional coaxial and in-plane magnetically coupled rings also offer some degree of bent powering path. However, they have not previously had enough mechanical flexibility to support the human motion, and may be difficult to synthesize and scale to large/curvy structures.

The present embodiments include development of metamaterial textiles that may be added to pre-existing clothing to create drag-and-drop near field multi-BANs. The present embodiments utilize flexible magneto-inductive elements that are tuned to the NFC band and support pick-and-placement of wireless nodes along the network. Inspired by modern low-cost vinyl clothing production, present approaches to integrating magneto-inductive networks on textiles eliminates the need for complicated sewing techniques and the relatively expensive conductive threads used to in such methods. This network may be designed, built and expanded at will to fit user needs. In many embodiments, the magneto-inductive elements may be composed of discrete planar and flexible microelectronics-free loops, with spectral behavior that is stabilized against human body effects. They thus create a tunable power/communication path across the human body. This is achieved by bounding the electric fields (which are easily perturbed by the effect of the human body) within the structure internally and utilizing quasi-static magnetic fields. A time-division, multiple access protocol may also be implemented to interrogate multiple NFC-enabled sensors connected across the body through discrete pieces of clothing.

The present embodiments create a secure on-demand BAN, whose communication link may span across different pieces of clothing, objects, or people. The battery-free and energy harvesting transponders used in the present embodiments may reduce user burden, allow continuous monitoring, and minimize node size (via battery elimination and memory reduction). In addition, the present embodiments may be used for self-sustained, zero-battery BAN ecosystems with cloud assistance.

In a first aspect, a body area network is provided, the body area network comprising: a first array of magnetically coupled resonators configured to propagate magneto-inductive (MI) surface waves; wherein the first array of magnetically coupled resonators comprises a plurality of MI elements; and wherein the first array of magnetically coupled resonators creates a flexible magnetic metamaterial path for wireless communication using the MI surface waves.

In an embodiment of the first aspect, the plurality of MI elements is tuned to near field communication (NFC) bandwidths.

In another embodiment of the first aspect, the first array of magnetically coupled resonators is configured to generate NFC-based MI surface waves.

In another embodiment of the first aspect, the body area network utilizes pairing and security of NFC protocol.

In another embodiment of the first aspect, the body area network further comprises at least one NFC enabled sensor connected to the first array of magnetically coupled resonators.

In another embodiment of the first aspect, a time-division, multiple access protocol is implemented to interrogate the at least one NFC enabled sensor.

In another embodiment of the first aspect, the body area network further comprises an NFC enabled reader.

In another embodiment of the first aspect, the NFC enabled reader is a mobile device.

In another embodiment of the first aspect, each of the plurality of MI elements comprises a flexible planar coil.

In another embodiment of the first aspect, the flexible planar coil is cut out of copper.

In another embodiment of the first aspect, each of the plurality of MI elements comprises a ground layer that minimizes spectral uncertainly due to a human body's parasitic effect.

In another embodiment of the first aspect, the ground layer includes at least one slot perpendicular to the flexible planar coil.

In another embodiment of the first aspect, the ground layer is cut out of aluminum foil.

In another embodiment of the first aspect, the first array of magnetically coupled resonators is integrated into a first clothing textile by stacking the flexible planar coil on the ground layer and adhering the ground layer to the first clothing textile using a heat press process.

In another embodiment of the first aspect, the heat press process includes applying heat to at least one heat transfer vinyl.

In another embodiment of the first aspect, the body area network further comprises a second array of magnetically coupled resonators, wherein the second array of magnetically coupled resonators comprises a plurality of MI elements.

In another embodiment of the first aspect, the first array of magnetically coupled resonators is integrated into a first clothing textile and the second array of magnetically couple resonators is integrated into a second clothing textile.

In another embodiment of the first aspect, the first array of magnetically coupled resonators and the second array of magnetically coupled resonators are separated by a clothing transition between the first clothing textile and the second clothing textile.

In another embodiment of the first aspect, the MI surface waves generate a wireless communication link across the clothing transition.

In another embodiment of the first aspect, the MI surface waves generate a wireless communication link between the first array of magnetically coupled resonators and at least one object.

In another embodiment of the first aspect, the body area network is associated with a first person and the MI surface waves generate a wireless communication link between the body area network and an external array of magnetically coupled resonators associated with a second person.

In another embodiment of the first aspect, the plurality of MI elements is connected in series.

In another embodiment of the first aspect, the plurality of MI elements is connected in parallel.

### BRIEF DESCRIPTION OF THE DRAWINGS

The various embodiments of the present on-demand functionalized textiles for near field multi-body area networks now will be discussed in detail with an emphasis on highlighting the advantageous features. These embodiments depict the novel and non-obvious features of on-demand functionalized textiles for near field multi-body area networks shown in the accompanying drawings, which are for illustrative purposes only. These drawings include the following figures:

FIG. 1a is a schematic diagram illustrating a planar magnet resonator in accordance with an embodiment of the invention.

FIG. 1b are graphs illustrating a ground layer minimizing spectral uncertainty due to a human body's parasitic effect in accordance with an embodiment of the invention.

FIG. 1c is a schematic diagram illustrating NFC sensors that may be dragged and dropped across magnetically coupled resonators with a horizontal distance (in x-direction) and a vertical distance in other directions in accordance with an embodiment of the invention.

FIG. 1d illustrates an equivalent circuit of the magneto-inductive (MI) metamaterial with potential object-to-object transitions in accordance with an embodiment of the invention.

FIG. 1e are dispersion diagrams for an array of resonators for various magnetic coupling coefficients in accordance with an embodiment of the invention.

FIG. 1f are diagrams illustrating a reader and multiple sensors utilized in an in-line serial and a T-shaped parallel array of resonators to form various signal paths around a body in accordance with an embodiment of the invention.

FIG. 1g is a diagram illustrating a metamaterial network (may also be referred to as “body area network” or “BAN”) streamlined into separate clothing pieces in accordance with an embodiment of the invention.

FIG. 2a is a graph illustrating a transmission profile of an inline network of resonators for varying inter-coil coupling (coil distancing) in accordance with an embodiment of the invention.

FIG. 2b is a graph illustrating a transmission profile for inter-coil (horizontal) coupling versus a coupling anomaly generated by a vertical distance (VD) in the middle of the network in accordance with an embodiment of the invention.

FIG. 2c is graph illustrating experimentally measured |S₂₁| over an inline array of resonators for various coil distancing (without VD), demonstrating the artificially-created standing wave along a magneto-inductive waveguide in accordance with an embodiment of the invention.

FIG. 2d is a diagram illustrating a side view of a magnetic field's distribution showing the standing wave (at βd_{c}≈π/2) along an inline resonator path with 2 cm of vertical distance in between (scalebar 10 cm) in accordance with an embodiment of the invention.

FIG. 2e is a diagram illustrating a top view of a magnetic field of a meandered S-shaped pathway in accordance with an embodiment of the invention.

FIG. 2f is a diagram illustrating a magnetic field of a circular pathway in accordance with an embodiment of the invention.

FIG. 2g is a diagram illustrating a magnetic field of a branched T-shaped pathway in accordance with an embodiment of the invention.

FIG. 2h are diagrams illustrating multiple coils sharing an intersection that may be merged into one piece to lower the system loss by reducing the number of interconnections, thus enhancing the transmission profile in accordance with an embodiment of the invention.

FIG. 3a are schematic diagrams illustrating resonator fabrication steps in accordance with an embodiment of the invention.

FIG. 3b is a diagram illustrating a close up image of a resonator stack in accordance with an embodiment of the invention.

FIG. 3c is diagram illustrating various implementations of flexible resonators for optimal signal transmission and power division (scalebar 10 cm) in accordance with an embodiment of the invention.

FIG. 3d is diagram illustrating a wearable modular network integrated into clothing and covered by transparent heat transfer vinyl as a mechanical fixture in accordance with an embodiment of the invention.

FIG. 3e is a diagram illustrating resonators that may be designed to cover wider nearfield areas and embedded underneath colored special vinyl designs in accordance with an embodiment of the invention.

FIG. 3f is a diagram illustrating battery-free NFC transponders integrated with strain and temperature sensors transferring respective sensor status to an NFC reader in accordance with an embodiment of the invention.

FIG. 4a illustrates flexible, drag-and-drop NFC networks on textiles including spectral stability of clothing integrated pathways under mechanical deformation in accordance with an embodiment of the invention.

FIG. 4b is a diagram illustrating averaged NFC PRR measured for various numbers of sensor-transponders in accordance with an embodiment of the invention.

FIG. 4c is a diagram illustrating PRR for 6 NFC transponders with various sampling rates in accordance with an embodiment of the invention.

FIG. 4d is a diagram illustrating packet loss for a moving sensor dragged under various velocities in accordance with an embodiment of the invention.

FIG. 5a is a diagram illustrating a body area network (BAN) utilizing multi-transponder and multi-BAN communication by textile-integrated waveguides on a shirt and pants and the pants/shirt terminals in accordance with an embodiment of the invention.

FIG. 5b illustrates real time short-term and low-speed monitoring of human activity realized by time-division based multiple sensor readout within the BAN in accordance with an embodiment of the invention.

FIG. 5c illustrates a high speed, long-term indoor walk/running activity measurement in accordance with an embodiment of the invention.

FIG. 5d illustrates monitoring of sensors during indoor running under various velocity profiles in accordance with an embodiment of the invention.

FIG. 5e illustrates long-term packet loss monitoring during indoor running in accordance with an embodiment of the invention.

FIG. 5f is a diagram illustrating body-to-body communication enabled by NFC's plug-and-play characteristics in accordance with an embodiment of the invention.

FIG. 6a is a diagram illustrating a large loop designed to resonate at 13.56 MHz without human's lossy skin effect, and a small loop designed to resonate at the same frequency using the ground layers with various slot distances in accordance with an embodiment of the invention.

FIG. 6b is a diagram illustrating a smaller coil integrated with ground layers of different slot gaps in accordance with an embodiment of the invention.

FIG. 6c is a graph illustrating benchtop measurements of a resonator's spectral characteristics in accordance with an embodiment of the invention.

FIG. 7a are graphs illustrating larger ohmic loss increases the attenuation (plots generated for k_{RR}=0.07) in accordance with an embodiment of the invention.

FIG. 7b are graphs illustrating a comparison of various standing wave modes which depend on the network's boundary condition and/or number of elements including resonator, reader, and sensors (plots generated for R_{R}=3) in accordance with an embodiment of the invention.

FIG. 8 is a schematic diagram illustrating coil geometry and a background partial ground in accordance with an embodiment of the invention.

FIG. 9 illustrates deriving an equivalent circuit of a coil array using Thevenin's equivalent circuit in accordance with an embodiment of the invention.

FIG. 10a is a schematic diagram illustrating a coupling anomaly along the network in accordance with an embodiment of the invention.

FIG. 10b are diagrams illustrating transmission coefficient (S₂₁) versus inter-relay and vertically distanced coupling factors in accordance with an embodiment of the invention.

FIG. 10c are diagrams illustrating coupling factor profile versus frequency for an inline network of 11 elements in accordance with an embodiment of the invention.

FIG. 10d is a graph illustrating benchtop transmission measurement showing the VD (in z-axis) can be compensated by reducing the neighbor coil distancing (increasing overlap in x-axis) to maintain the same level of transmission in accordance with an embodiment of the invention.

FIG. 11a illustrates coupling profiles with similar VD placements from either end of the linear array being identical in accordance with an embodiment of the invention.

FIG. 11b illustrates similar reciprocity when two VDs occur along the network in accordance with an embodiment of the invention.

FIG. 12 illustrates ex vivo transmission per element and versus Tx/Rx distance obtained from an I-shaped network in accordance with an embodiment of the invention.

FIG. 13a illustrates electrical characterization by FEM simulation and measurements for various grounded resonators along with the magnetic field intensity profile in accordance with an embodiment of the invention.

FIG. 13b illustrates experimentally measured S-parameter matrixes (at 13.56 MHz) for resonators as waveguides and/or power splitters in accordance with an embodiment of the invention.

FIG. 13c illustrates experimentally measured transmission profile comparison for straight, branched, and closed-loop architectures in accordance with an embodiment of the invention.

FIG. 14a are diagrams illustrating an inline architecture in accordance with an embodiment of the invention.

FIG. 14b are diagrams illustrating a meandered architecture in accordance with an embodiment of the invention.

FIG. 15a illustrates various shirt networks enabled by complex parallel and closed loop metamaterial paths in accordance with an embodiment of the invention.

FIG. 15b illustrates a colored vinyl implementation that may be utilized to appear next to the network in accordance with an embodiment of the invention.

FIG. 15c illustrates a colored vinyl implementation that may be utilized to conceal the network underneath in accordance with an embodiment of the invention.

FIG. 16 is a diagram illustrating a board design of an NFC transponder in accordance with an embodiment of the invention.

FIG. 17 is a flowchart illustrating software based TDMA and sensor data processing in accordance with an embodiment of the invention.

FIG. 18a illustrates full body movement detection in accordance with an embodiment of the invention.

FIG. 18b illustrates lower body movement detection in accordance with an embodiment of the invention.

FIG. 18c illustrates lower body cycling detection in accordance with an embodiment of the invention.

FIG. 18d illustrates test of a network's compatibility with off-the-shelf sensor and compatible (e.g., Android application) in accordance with an embodiment of the invention.

FIG. 19a illustrates frequency and magnitude shifts caused by the high permittivity and the loss of added water in accordance with an embodiment of the invention.

FIG. 19b illustrates robustness of magneto-inductive BAN's spectral characteristics during different phases of washing in accordance with an embodiment of the invention.

### DETAILED DESCRIPTION OF THE DRAWINGS

The following detailed description describes the present embodiments with reference to the drawings. In the drawings, reference numbers label elements of the present embodiments. These reference numbers are reproduced below in connection with the discussion of the corresponding drawing features.

Turning now to the drawings, on-demand functionalized textiles for near field multi-body area networks are further described below. Wearable and implantable sensors may be linked together to create multi-node wireless networks that may be used in the development of advanced healthcare monitoring technologies. Such body area networks may utilize secure, seamless, and versatile communication links that may operate across the complex human body, but may suffer from short ranges, low power, or the need for direction connection links. In many embodiments, the present embodiments illustrate that textile-integrated metamaterials may be used to drive long-distance near-field communication (NFC)-based magneto-inductive waves along and between multiple objects. In various embodiments, such metamaterials may be built from arrays of discrete, anisotropic magneto-inductive (MI) elements, and create mechanically-flexible systems capable of battery-free communication among NFC-enabled devices that may be placed anywhere close to networks. The present embodiments offer a secure and on-demand body are network that exhibits complex architectures and straightforward expansion that may span across different pieces of clothing, objects, and/or people.

As further described below, the present embodiments demonstrate a near field multi-BAN in which wireless sensing nodes may be placed anywhere along the network in a plug-and-play fashion, and the network itself may be designed, built and extended with minimal effort. Further, these networks may offer secure and battery-free, object-to-object and/or body-to-body transfer of near-field communication. The networks may be created using a low-cost, rapid prototyping technique of synthesizing textiles functionalized with magneto-inductive waveguides tuned to NFC bands. In several embodiments, the modular nature of these networks may allow the present embodiments to be extended in a number of directions. The layer-by-layer conductive vinyl may be readily modified with emerging bio-interactive materials and sensors, and could allow electronic components to be built alongside the body. In addition, the magneto-inductive elements may be scalable to different frequency bands as required by a particular application and/or the user, enabling application or person-specific transmission. Network design and analysis in accordance with embodiments of the invention are further discussed below.

### Network Design and Analysis

Magneto-inductive waves may propagate through an array of magnetically coupled resonant structures that possess equivalent spectral characteristics. The resonators may be implemented in different forms depending on the network's desired characteristics. Traditionally, they are created with rigid loops whose impedance may be tuned by lumped elements.

In the present embodiments, the requirements of BANs may impose a narrow set of constraints on the performance of the magneto-inductive waveguides, as it typically should exhibit high degrees of flexibility, be insensitive to bodily motion, be easy to extend, and possess a microelectronic-free design. In many embodiments, multi-turn flexible planar coils made of metal (e.g., aluminum and/or copper) foils as resonators may be integrated into the clothing textile. The resonance characteristic of such coils, however, may depend highly on the undesired parasitic capacitances between the coil and human body. This may interfere with the magneto-inductive wave propagation as the textile integrated network is not fixed on the body and the distance from the skin (dₛₖᵢₙ) may move slightly during routine activities even on tightly-fitted clothing. To eliminate this effect, the coil may be stacked on a ground shield layer to suppress the inductor's electric field from entering the body. This approach may add considerable intrinsic capacitance to the resonator (C_{R}) and shift down the resonant frequency, which reduces the loop's length and thus enhances the ohmic loss and self-inductance of the resonator (R_{R} and L_{R}, respectively). However, this ground layer may be subject to inducing eddy currents due to proximity to the loop traces.

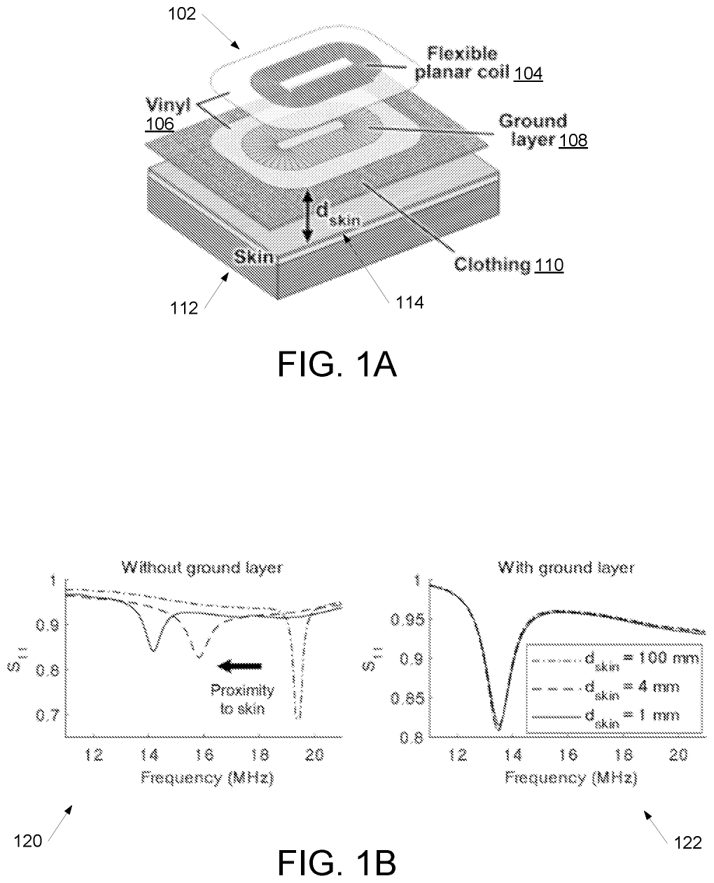

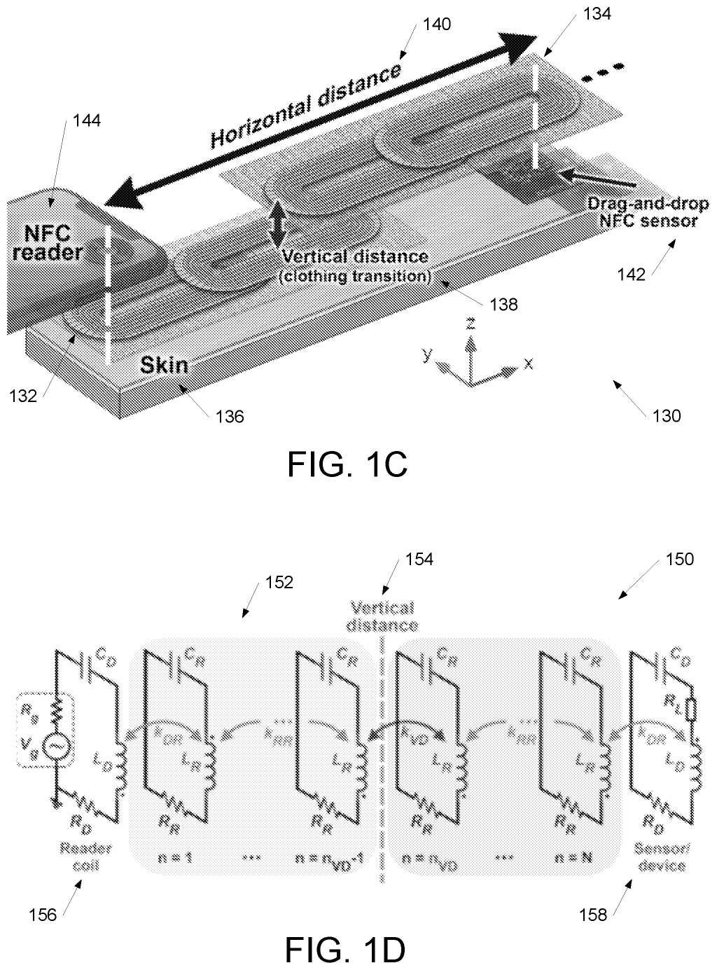

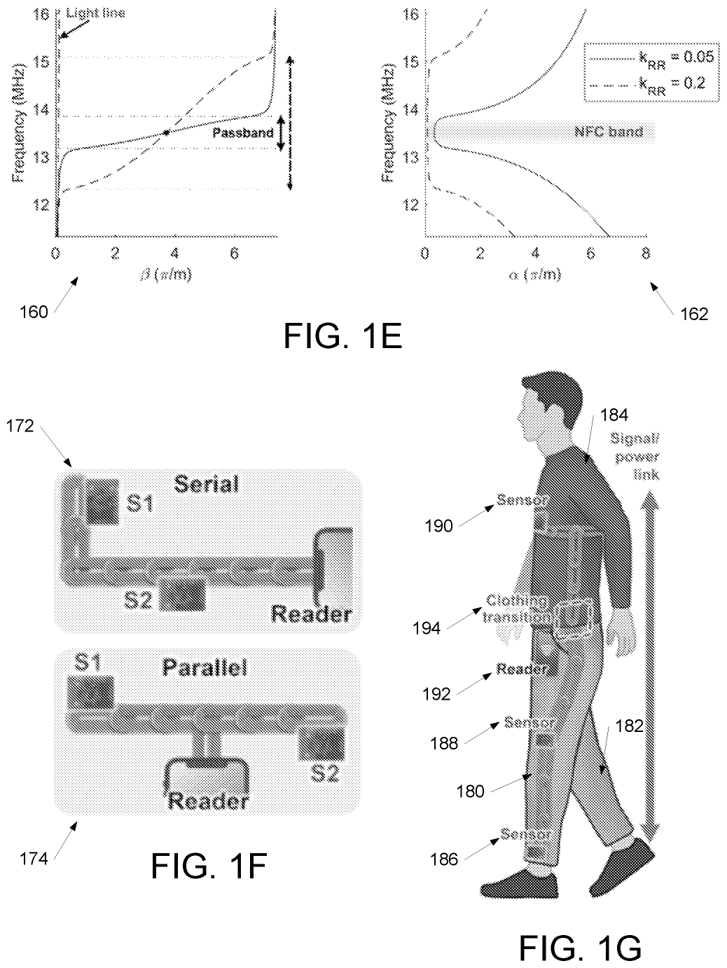

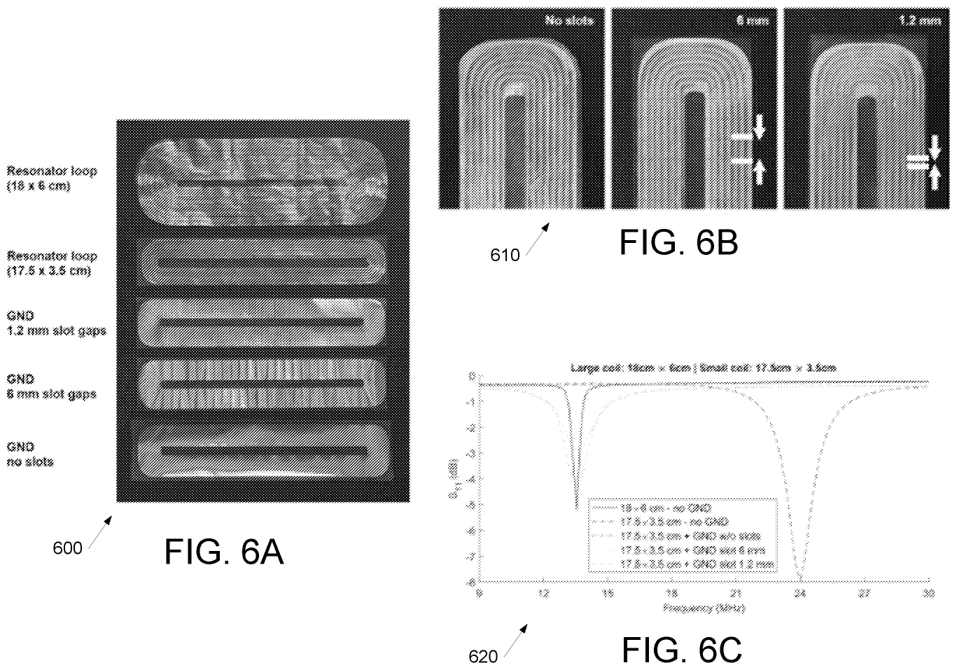

To avoid significant loss of the resonator's quality factor, the present embodiments includes several slots on the ground layer perpendicular to the loop traces to terminate the eddy currents. A schematic diagram illustrating a planar magnet resonator in accordance with an embodiment of the invention is shown in FIG. 1a. The planar magnet resonator 102 may include a flexible planar coil 104 and a ground layer 108. In some embodiments, the flexible planar coil 104 and/or the ground layer 108 may be on vinyl 106. The planar magnet resonator 102 may be placed on top of clothing 110 that may be on a person's skin 112. The distance 114 between the skin and the resonator 102 is shown. Graphs 150, 160 illustrating a ground layer minimizing spectral uncertainty due to a human body's parasitic effect in accordance with an embodiment of the invention is shown in FIG. 1b. Specifically, graph 120 is without ground layer and graph 122 is with a ground layer. In many embodiments, this may compensate for the power dissipation generated from the flow of image currents on the ground layer. Therefore, the slotted ground layer may intervene in between the loop and skin, eliminate the unpredicted spectral shift of the resonator, and help to miniaturize the loop while not significantly affecting the resonator's quality factor compared to when the loop is directly put on the skin (see FIG. 6). FIG. 6 illustrates a ground (GND) layer's slot effect on a resonator's spectral properties. A diagram 600 illustrating a large loop designed to resonate at 13.56 MHz without human's lossy skin effect, and a small loop designed to resonate at the same frequency using the ground layers with various slot distances in accordance with an embodiment of the invention is shown in FIG. 6a. A diagram 610 illustrating a smaller coil integrated with ground layers of different slot gaps in accordance with an embodiment of the invention is shown in FIG. 6b. A graph 620 illustrating benchtop measurements of a resonator's spectral characteristics in accordance with an embodiment of the invention is shown in FIG. 6c. As will be discussed further below, the magnetic field in this scenario may still be allowed to flow below and above the resonator despite this ground layer.

The magneto-inductive waves can propagate through more convoluted pathways involving arrays of magnetically coupled resonators. A schematic diagram illustrating NFC sensors that may be dragged and dropped across the magnetically coupled resonators with a horizontal distance (in x-direction) and a vertical distance in other directions in accordance with an embodiment of the invention is shown in FIG. 1c. Diagram 130 includes magnetically coupled resonators 132, 134, on skin 136, having a horizontal distance 140 and a vertical distance 138. The NFC sensor(s) 142 may be dragged and dropped across the magnetically coupled resonators 132, 134. In various embodiments, this magnetic connection allows for more flexibility in terms of the resonators' 132, 134 relative placements and introduces a horizontal distance 140 within a network between the NFC reader 144 and sensor nodes (in x-direction), in addition to the vertical distances 138 (VD) between two neighbor nodes (resonator/device) on different pieces of clothing (or z axis). Such networks show propagation behavior along the coils (x direction) and typical near field properties in other directions. Thus, the nodes (including reader and multiple sensors) in the close vicinity of the coil network would be magnetically connected. The network's equivalent circuit 150 comprised of N coupled coils 152 plus one reader 156 and sensor 158 with a vertical distance 154 in between is shown in FIG. 1d.

The present embodiments may assume that the current flowing in the n^{th} resonator has a sinusoidal time-dependency with an angular frequency of ω. Here, the resonator-coils each with an impedance of

$${Z}_{R} = {R}_{R} + j ω {L}_{R} + \frac{1}{j ω {C}_{R}} ,$$

may be inductively coupled to their closest neighbor resonator with the mutual coupling of M_{RR}=k_{RR}L_{R} where M and k represent the mutual inductance and coupling factor frequency respectively (index R_{R} shows inter-resonator relations). In many embodiments, the resonators form a linear array with an equal distancing of d_{c} between two neighbor coils. For simplicity, it may be assumed the vertical distance is ignorable (k_{VD}=k_{RR}). Further, the current running on the n^{th} resonator (ranging from 1 to N) in an array can be represented by:

$${I}_{n} = {I}_{1} {e}^{j {ϕ}_{1}} {e}^{- j γ ( n - 1 ) {d}_{c}} \qquad ( 1 )$$

where γ is the travelling wave's propagation constant, I₁ and ϕ₁ are the first loop's current magnitude and phase depending on the excitation (boundary conditions imposed by reader's V_{g}). The Kirchhoff's voltage law for the n^{th} coil follows:

$${Z}_{R} {I}_{n} + j ω {M}_{RR} ( {I}_{n - 1} + {I}_{n + 1} ) = 0 \qquad ( 2 )$$

which leads to the dispersion equation:

$$γ = \frac{1}{{d}_{c}} \times {cos}^{- 1} ( \frac{- {Z}_{R}}{2 j ω {M}_{RR}} ) \qquad ( 3 )$$

This structure may support forward and backward traveling waves and thus form a standing wave along the resonators array. To match the standing wave's spatial harmonics with the array's geometry, we define γₘ=4γ/m where m (>1) indicates the number of coils between two spatially equal-phase planes along the standing wave. This enables analyzing the propagation characteristics per unit of resonator (instead of length) and would be ultimately helpful to identify the resonator number on which the standing wave's peak places. The per-resonator expression of the spatial harmonics facilitates the network's design for the end user to plan the number of resonators on each piece of clothing and thus optimize the BAN. Here, γₘ=β−jα is the harmonic propagation constant (β as the phase and α as attenuation constants) and is calculated for our typical resonator properties and shown in FIG. 1e. Dispersion diagrams 160, 162 for an array of resonators for various magnetic coupling coefficients in accordance with an embodiment of the invention is illustrated in FIG. 1e. The lower and higher cutoff frequencies (MI wave passband) are marked by dotted lines and specify the bandwidth. The light line (with a large slope of the light velocity in free space) is shown by the dashed line.

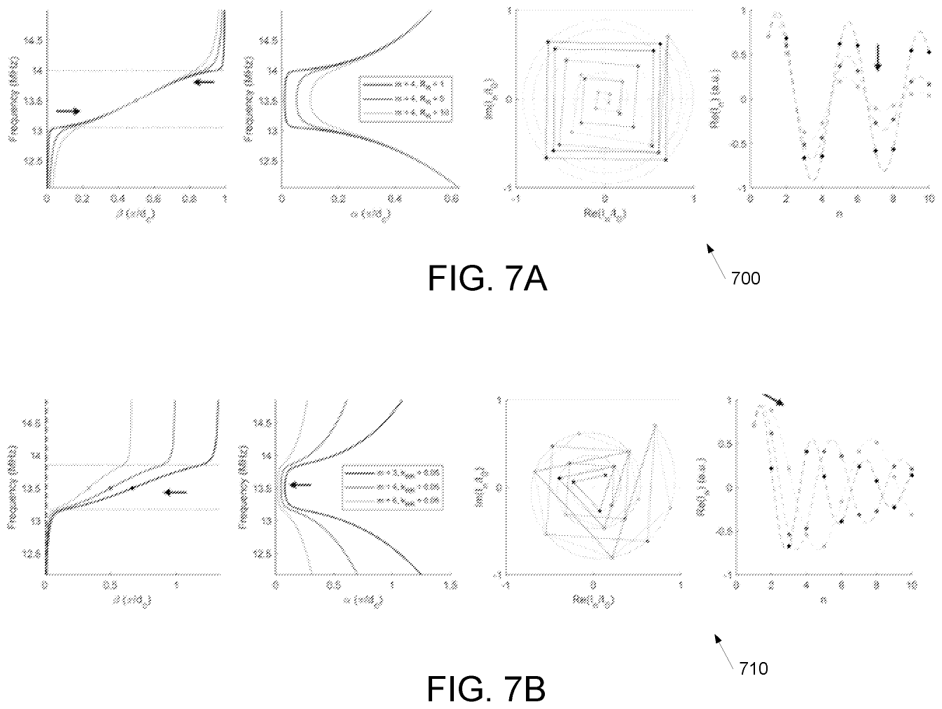

The propagation constant profile β(ω) may possess different values depending on the coil geometry (reflected in Z_{R}), coupling factor, and harmonic modes (m, which is not necessarily an integer). Dispersion profiles for various modes and electrical properties are compared in FIG. 7. Graphs 710 illustrating larger ohmic loss increases the attenuation (plots generated for k_{RR}=0.07) in accordance with an embodiment of the invention is shown in FIG. 7a. Graphs 720 illustrating a comparison of various standing wave modes which depend on the network's boundary condition and/or number of elements including resonator, reader, and sensors (plots generated for R_{R}=3) in accordance with an embodiment of the invention are shown in FIG. 7b.

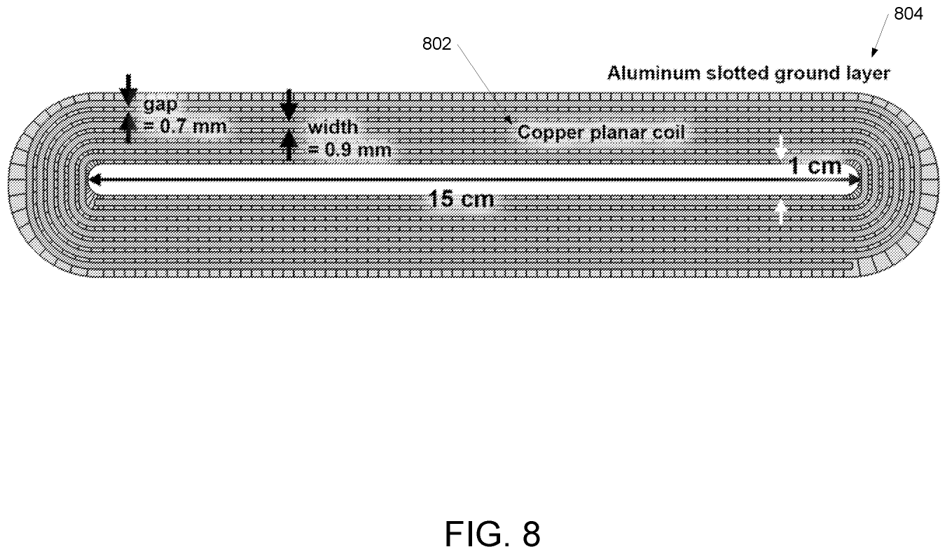

A schematic diagram illustrating coil geometry 802 and a background partial ground 804 in accordance with an embodiment of the invention is shown in FIG. 8. The coils were designed and tuned (detailed properties shown in FIG. 8) to resonate at NFC's standard frequency (13.56 MHz). To afford enough bandwidth for the amplitude shift keying utilized in NFC protocols, the auxiliary carriers (distanced 848 kHz from the main carrier at 13.56 MHZ) may be covered by the metamaterial's passband. As shown in FIG. 1e, this may be tuned by the inter-resonator coupling (k_{RR}) or equivalently the neighbor coil distancing (d_{c}). The passband calculations obtained from the dispersion diagrams show that a k_{RR} value of 0.1 is capable of providing sufficient bandwidth for successful long-term communication under varying mechanical distress. This is approximately equivalent to less than 20% neighbor coil overlap (3.2 cm for our typical rectangular loop that possesses a length of 17 cm). Unlike traditional BANs that utilize coils connected by wire, in the present embodiments, the inter-resonator magnetic coupling enables complex network architectures with user-friendly extensions such as inline or fork connections. Diagrams illustrating a reader and multiple sensors utilized in an in-line serial 172 and a T-shaped parallel 174 array of resonators to form various signal paths around a body in accordance with an embodiment of the invention are shown in FIG. 1f. When integrated into clothing, it may allow for the BAN's complex signal paths to span across multiple layers of disconnected clothing (e.g. from pants to shirts), distinguishing itself from other textile-BANs that rely on a wire- or conductive thread-based connection. A diagram illustrating a metamaterial network 180 streamlined into separate clothing pieces in accordance with an embodiment of the invention is shown in FIG. 1g. The metamaterial network 180 may be easily streamlined into separate clothing pieces (e.g., pants 182 and shirt 184 having a clothing transition 194), enabling high flexibility necessary for daily routines and significant horizontal range extension. The metamaterial network 180 may include various components such as, but not limited to, sensor(s) 186, 188, 190 and reader(s) 192. The nodes may be placed anywhere close to (within a few centimeters of) any point of the network.

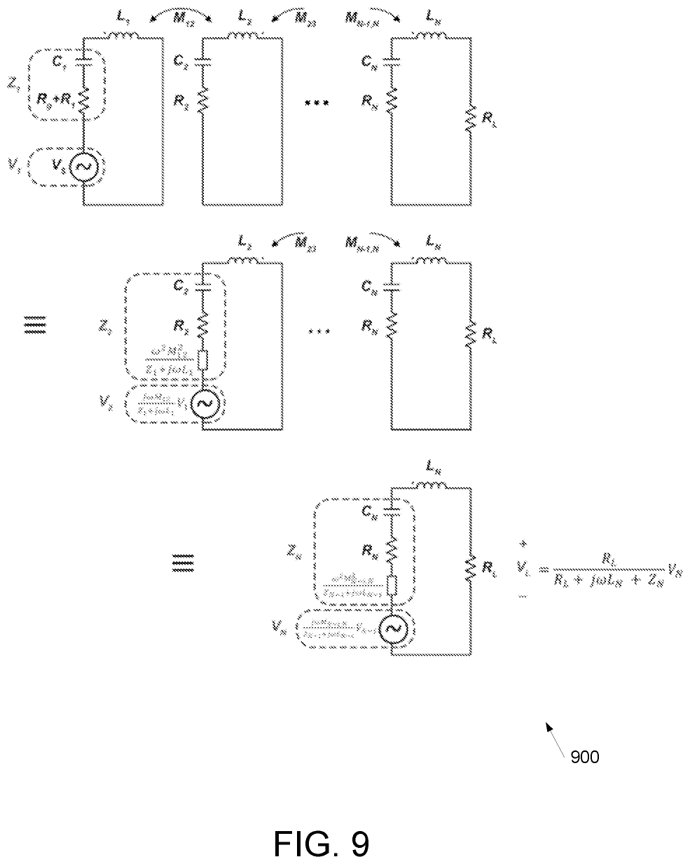

The VD's effect can be evaluated using the circuit theory to calculate the efficiency of the network (see FIG. 9 for deriving the equivalent circuit of the resonator chain). For example, deriving an equivalent circuit 900 of a coil array using Thevenin's equivalent circuit in accordance with an embodiment of the invention is shown in FIG. 9. The mutual inductances may take different values depending on if there is a vertical distance along the chain.

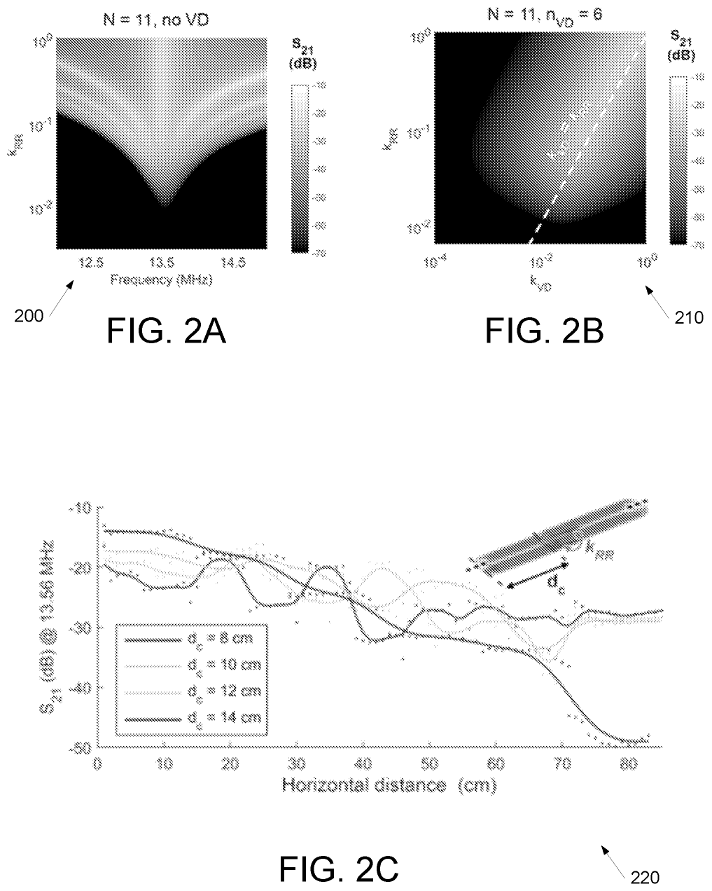

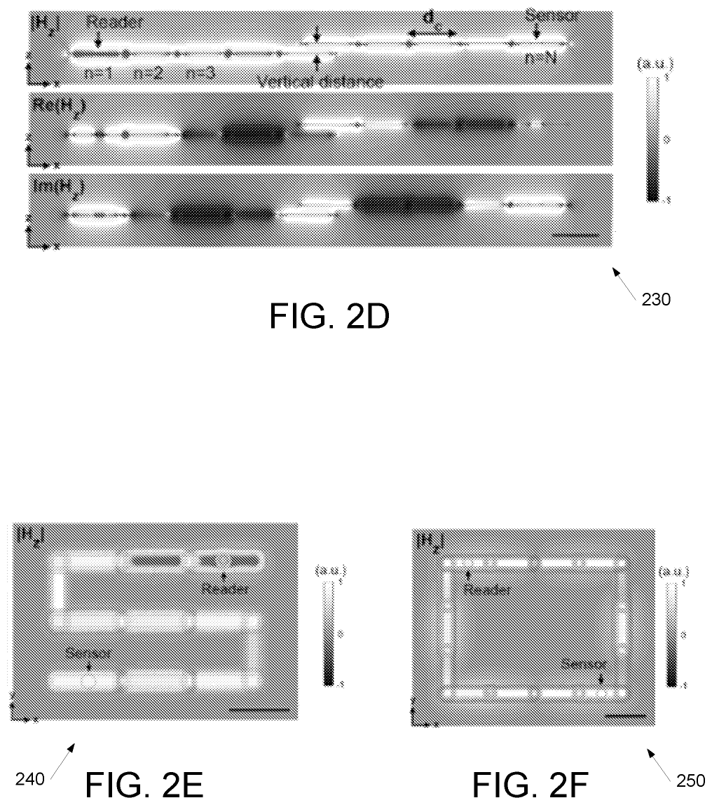

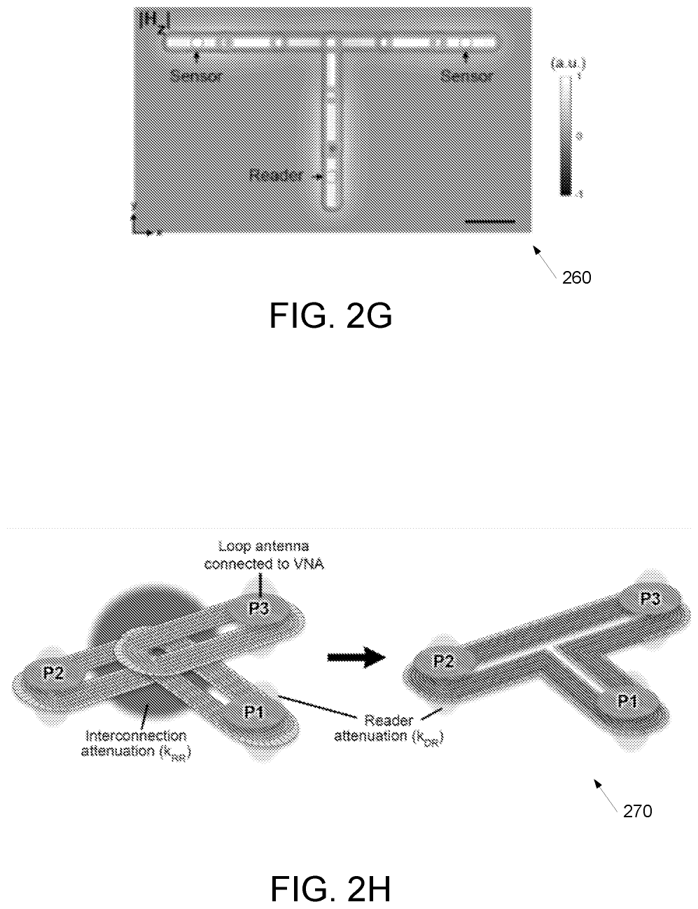

A graph 200 illustrating a transmission profile of an inline network of resonators for varying inter-coil coupling (coil distancing) in accordance with an embodiment of the invention is shown in FIG. 2a. First, the transmission (S₂₁) profile of a network (with N=11) without VD is shown in FIG. 2a where the reader (Tx) and sensor (Rx) at the ends of the inline resonator chain are connected to the first and second ports, respectively. For large enough k_{RR} values, the peak S₂₁ (originally centered at 13.56 MHz) splits into N resonances, correlating to the metamaterial passband. This aligns with what is referred to as the strong coupling of magnetic resonances.

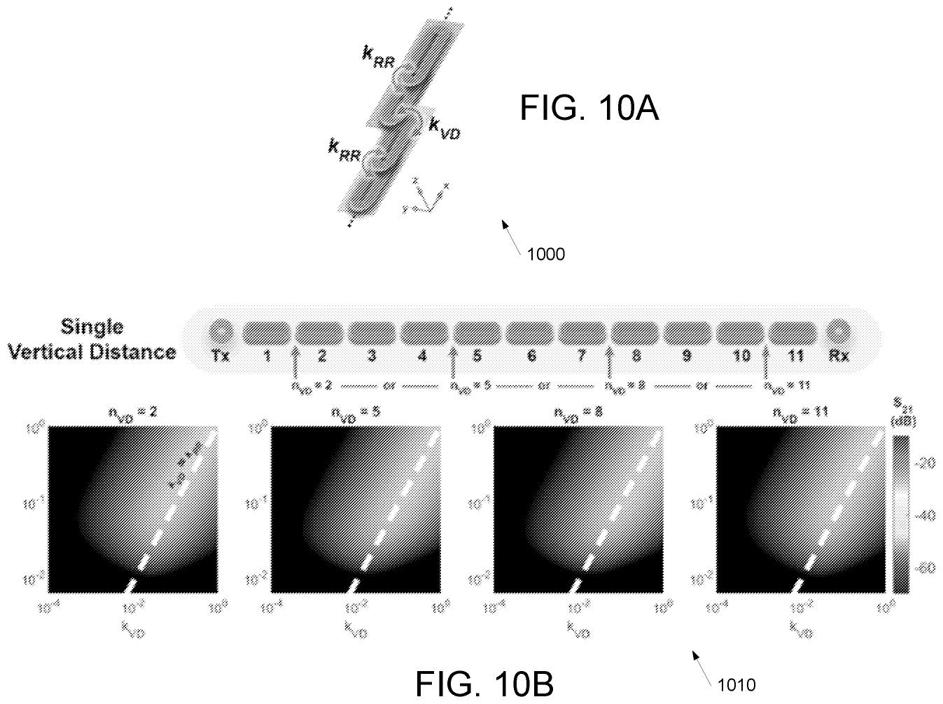

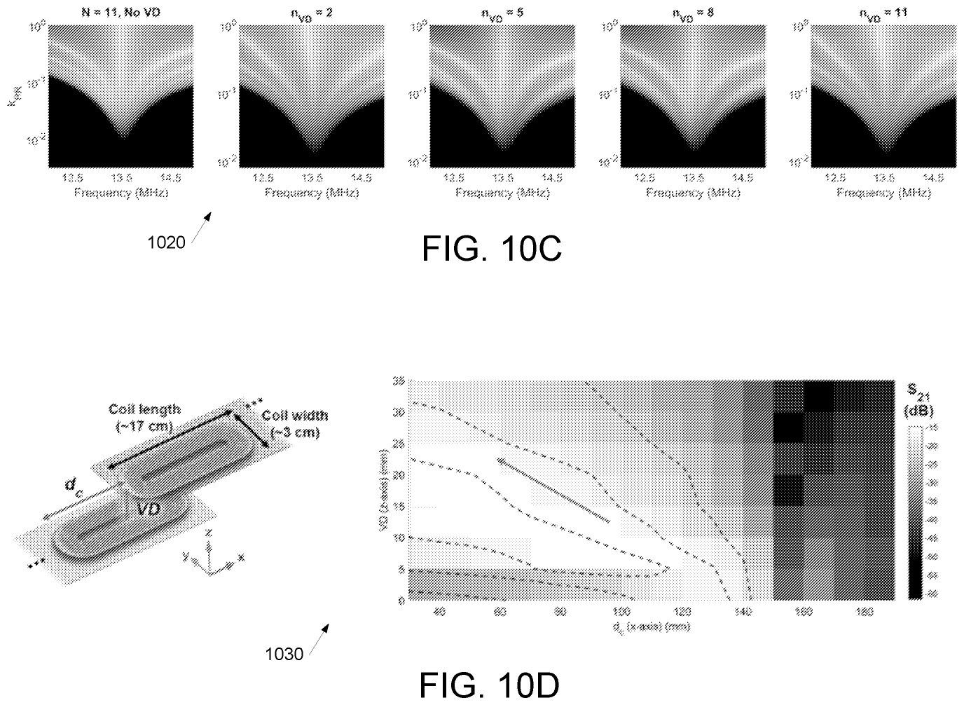

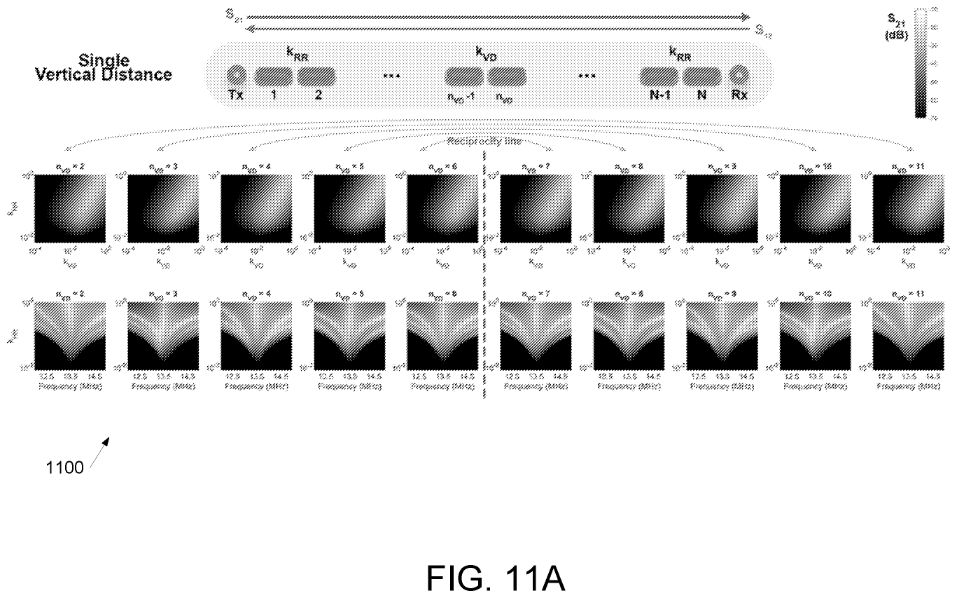

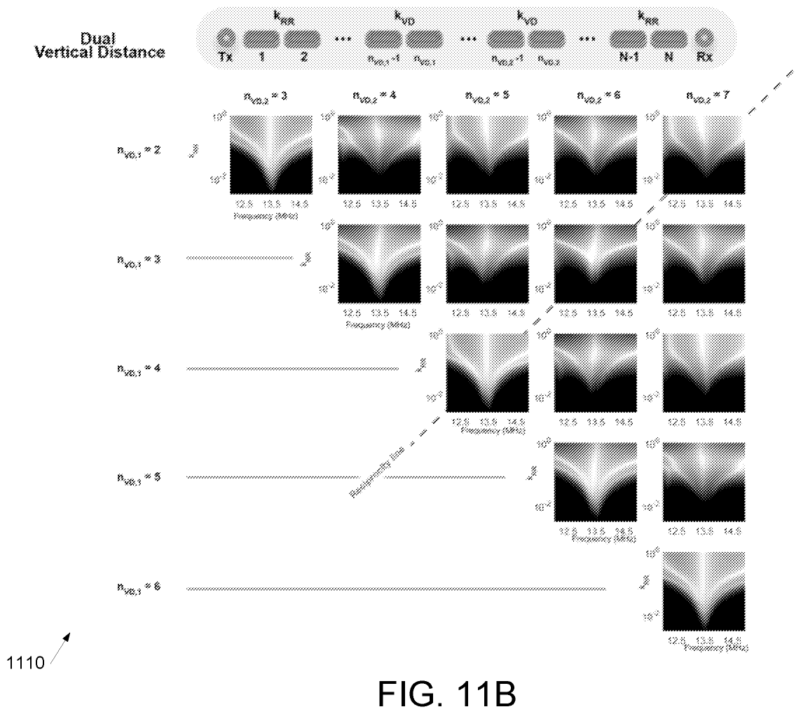

A graph 210 illustrating a transmission profile for inter-coil (horizontal) coupling versus a coupling anomaly generated by a vertical distance (VD) in the middle of the network in accordance with an embodiment of the invention is shown in FIG. 2b. Adding a VD with the coupling factor of k_{VD} at the 6^{th} resonator (middle of the chain) may affect the transmission at the central frequency (see FIG. 2b). It is shown, however, that the network demonstrates peak transmission performance when k_{VD}=k_{RR}. To operate close to this constraint, one may compensate for the low k_{VD} (due to the z-axis distance) by increasing the coil overlap (along the x-axis). The S₂₁ profile comparison for various VD placements, and modulating VD compensation is shown in FIG. 10. A schematic diagram 1000 illustrating a coupling anomaly along the network in accordance with an embodiment of the invention is shown in FIG. 10a. Diagrams 1010 illustrating transmission coefficient (S₂₁) versus inter-relay and vertically distanced coupling factors in accordance with an embodiment of the invention are shown in FIG. 10b. Diagrams 1020 illustrating coupling factor profile versus frequency for an inline network of 11 elements in accordance with an embodiment of the invention are shown in FIG. 10c. A diagram 1030 illustrating benchtop transmission measurement (at 13.56 MHz) showing the VD (in z-axis) can be compensated by reducing the neighbor coil distancing (increasing overlap in x-axis) to maintain the same level of transmission (contours are shown by dashed lines) is shown in FIG. 10d. Cellulose sheets were used as a variable z-axis spacer to mimic the intervening clothing. Due to the reciprocal nature of the magneto-inductive array, various (single or multiple) VD placements along an inline network may possess similar transmission profiles (see FIG. 11).

FIG. 11 illustrates system receprocity for single and two vertical discontinuities. Coupling profiles 1100 with similar VD placements from either end of the linear array being identical in accordance with an embodiment of the invention is shown in FIG. 11a. In reference to FIG. 11a, due to the inline network's theoretical reciprocity, certain coupling profiles such as n_{VD}=(2,11) and (5,8) with similar VD placements from either end of the linear array are identical (here N=11). Similar reciprocity 1110 when two VDs occur along the network in accordance with an embodiment of the invention is shown in FIG. 11b. In reference to FIG. 11b, similar reciprocity can be found when two VDs occur along the network (here N=7). This ultimately simplifies spectral optimization and minimizes design rules by enabling planning of resonator placements prior to integration into the clothing.

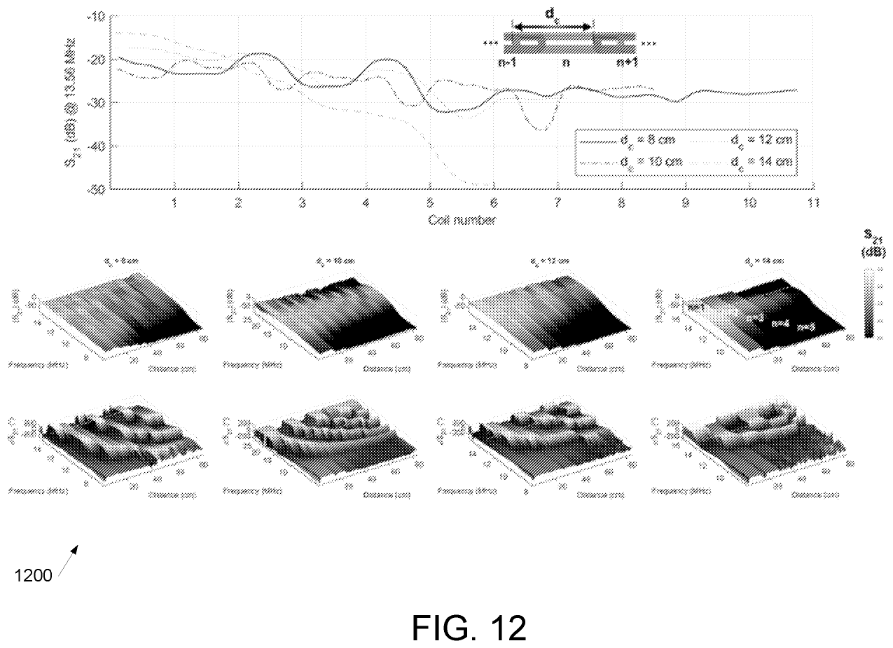

A graph 220 illustrating experimentally measured |S₂₁| over an inline array of resonators for various coil distancing (without VD), demonstrating the artificially-created standing wave along a magneto-inductive waveguide in accordance with an embodiment of the invention is shown in FIG. 2c. According to the benchtop measurement of the inline array of resonators with fixed Tx and a moving Rx, larger k_{RR} (or smaller d_{c}) results in enhanced horizontal range, but meanwhile, S₂₁ peak splits (as in FIG. 2a) and ends up with non-monotonic S₂₁ fluctuations along the network. The fluctuations agree well with the envelop of the Iₙ profile (shown in FIG. 7b) and shows standing wave formation. In addition, the wideband spectrum of the transmission (depicted in FIG. 12) demonstrates the ability to optimize the metamaterial's bandwidth. Diagrams 1200 illustrating an ex vivo transmission per element and versus Tx/Rx distance obtained from an I-shaped network in accordance with an embodiment of the invention is shown in FIG. 12. Additionally, small de may require a larger number of coils per unit of length, eventually increasing the loss at the coil transitions. Although long coils decrease the number of transitions, on the other hand, it may reduce the number of turns in each coil (to maintain the same resonance frequency) and lowers L_{R} and k_{RR}, which may not be ideal.

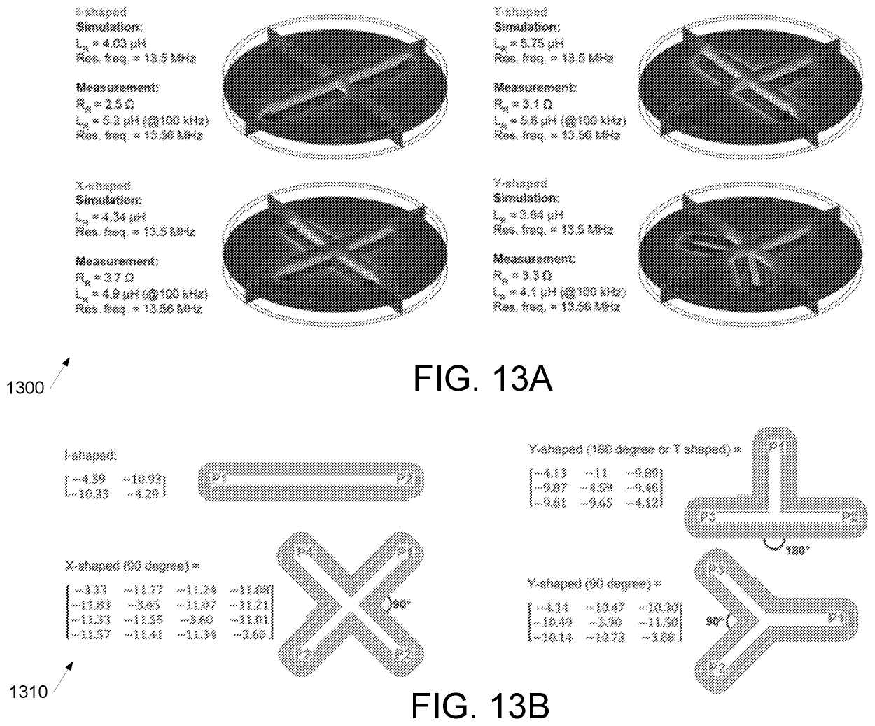

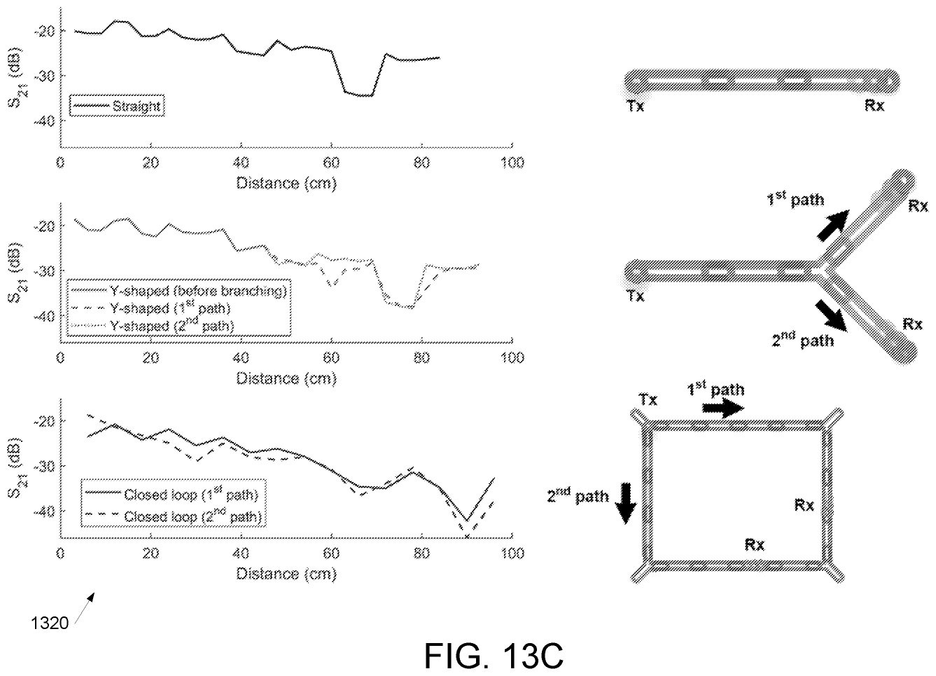

A diagram 230 illustrating a side view of a magnetic field's distribution showing the standing wave (at βd_{c}≈π/2) along an inline resonator path with 2 cm of vertical distance in between (scalebar 10 cm) in accordance with an embodiment of the invention is shown in FIG. 2d. The magnetic field profile simulation in an inline array (with a test VD in the middle) may be implemented by the finite element method (see FIG. 2d) and demonstrates the standing wave formation and the feasibility of the present methods for clothing transitions. Powerfully, the approach of magnetically coupled resonators offers in-plane coil rotation (in addition to bending), enabling the creation of various pathway styles including a meandered ‘S-shaped’ network (see FIG. 2e). This allows the magneto-inductive waveguide/network to pass through various points on the human body for sensing purposes. A diagram 240 illustrating a top view of a magnetic field of a meandered S-shaped pathway in accordance with an embodiment of the invention is shown in FIG. 2e. A diagram 250 illustrating a magnetic field of a circular pathway in accordance with an embodiment of the invention is shown in FIG. 2f. The open-ended network may be generalized to a closed-loop and allow for reduction of the total number of resonators in the BAN, although it may enforce additional boundary conditions on the network's dispersion and suppress some modes. This concept can be robustly expanded where a pathway branches off from a main signal path. A diagram 260 illustrating a magnetic field of a branched T-shaped pathway in accordance with an embodiment of the invention is shown in FIG. 2g. Each of the branches may be analyzed separately by considering that their first element's current is identical to the main pathway's last element. The experimental transmission profiles of closed-loop and branched network architectures (operating as an magneto-inductive power divider) are quantitatively compared to the straight, undivided pathway. This, in addition to the electrical measurements and simulation of various resonators are illustrated in FIG. 13. Diagrams 1300 illustrating benchtop measured and simulated spectral characteristics of the magneto-inductive elements all resonating at 13.56 MHz with various architectures. In reference to FIG. 13a electrical characterization by FEM simulation and measurements for various grounded resonators along with the magnetic field intensity profile in accordance with an embodiment of the invention is illustrated. Due to the distributed and fringing capacitances, the equivalent capacitance of each structure can be calculated from the self-inductance and resonance frequency (coils resonate at 13.56 MHz). Measurement values were substituted in the circuit model. Diagrams 1310 illustrating experimentally measured S-parameter matrixes (at 13.56 MHz) for resonators as waveguides and/or power splitters in accordance with an embodiment of the invention is shown in FIG. 13b. In reference to FIG. 13b experimentally measured S-parameter matrixes (at 13.56 MHz) for resonators as waveguides and/or power splitters (ground layer is not shown) is illustrated. Diagrams 1320 illustrating experimentally measured transmission profile comparison for straight, branched, and closed-loop architectures in accordance with an embodiment of the invention is shown in FIG. 13c. In reference to FIG. 13c experimentally measured transmission profile comparison for straight, branched, and closed-loop architectures is illustrated. The S-parameters here include the loss caused by the reader loop antenna's mutual coupling with the resonator ports (each reader antenna imposes about 2 dB of loss), thus the actual transmissions (Sᵢⱼ) are larger about 4 dB (we did not compensate for this to ensure the VNA's calibration accuracy).

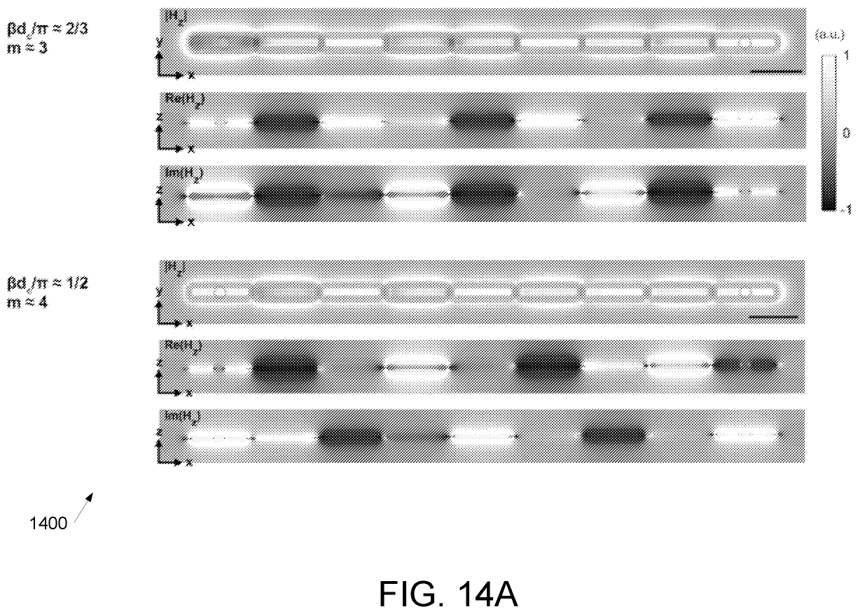

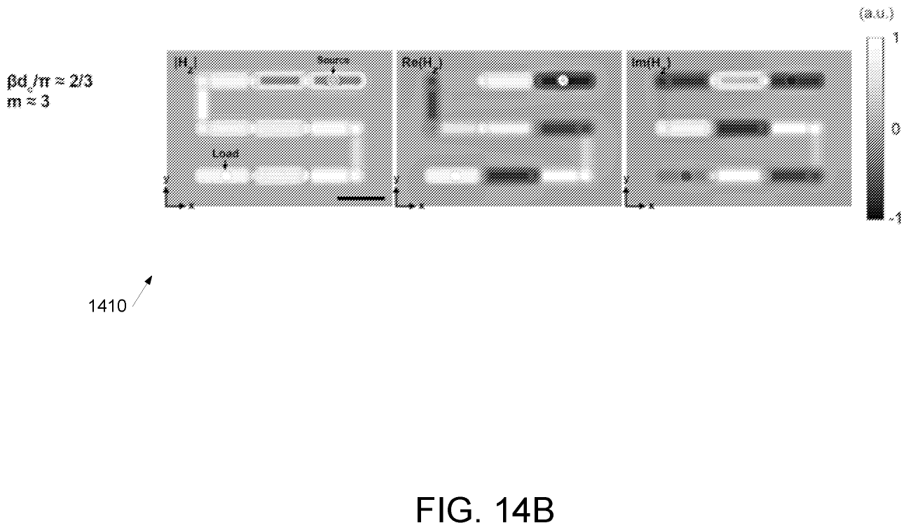

The magnetic profiles may be simulated for various modes (induced by variant coil distances) and shown in FIG. 14. Diagrams 1400 illustrating an inline architecture in accordance with an embodiment of the invention are shown in FIG. 14a. Here, m represents the number of coils inducing a phase shift of 2× on the standing wave (scalebar 10 cm). Diagrams 1410 illustrating a meandered architecture in accordance with an embodiment of the invention are shown in FIG. 14b. Here, m represents the number of coils inducing a phase shift of 2× on the standing wave (scalebar 10 cm).

To reduce the number of resonant elements, enhance the transmission, and achieve higher mechanical flexibility at the network's branched sections, the present embodiments may merge the intersection coils (e.g. three coils at the T-shaped junction) into one multi-ended coil. This noticeably reduces the system loss (created in majority by inter-resonators' coupling loss). The reader/network mutual coupling and resonator's ohmic loss, however, yet exist but in lower orders compared to the interconnection attenuations. Diagrams 270 illustrating multiple coils sharing an intersection that may be merged into one piece to lower the system loss by reducing the number of interconnections, thus enhancing the transmission profile in accordance with an embodiment of the invention are shown in FIG. 2h.

Although specific network designs and analysis are discussed above with respect to FIGS. 1a-2h and 6a-11, any of a variety of networks including a variety of resonators, readers, sensors, array configurations, MI metamaterials, metamaterial pathways, analysis, and communication protocols as appropriate to the requirements of a specific application can be utilized in accordance with embodiments of the invention. Clothing integration and multi-sensor studies in accordance with embodiments of the invention is discussed further below.

### Clothing Integration and Multi-Sensor Studies

An ideally-functional BAN should be adaptable to pre-existing clothing and readily expand to desired body areas based on the user's needs. These should possess mix-and-match features in terms of positioning of the network and multiple sensors. Additionally, their functionality should not be limited to only a few hot spots on which the reader/sensor can be placed. Conventional textile-BANs, however, usually use conductive thread sewed on the clothing, and are not particularly affordable or easy to fabricate. In addition, these textile networks usually suffer from the inability to cross different pieces of clothing due to their wired nature. Inspired by modern low-cost, vinyl heat-transfer designs, the present embodiments address such needs by utilizing a facile and versatile technique of integrating metamaterial railways.

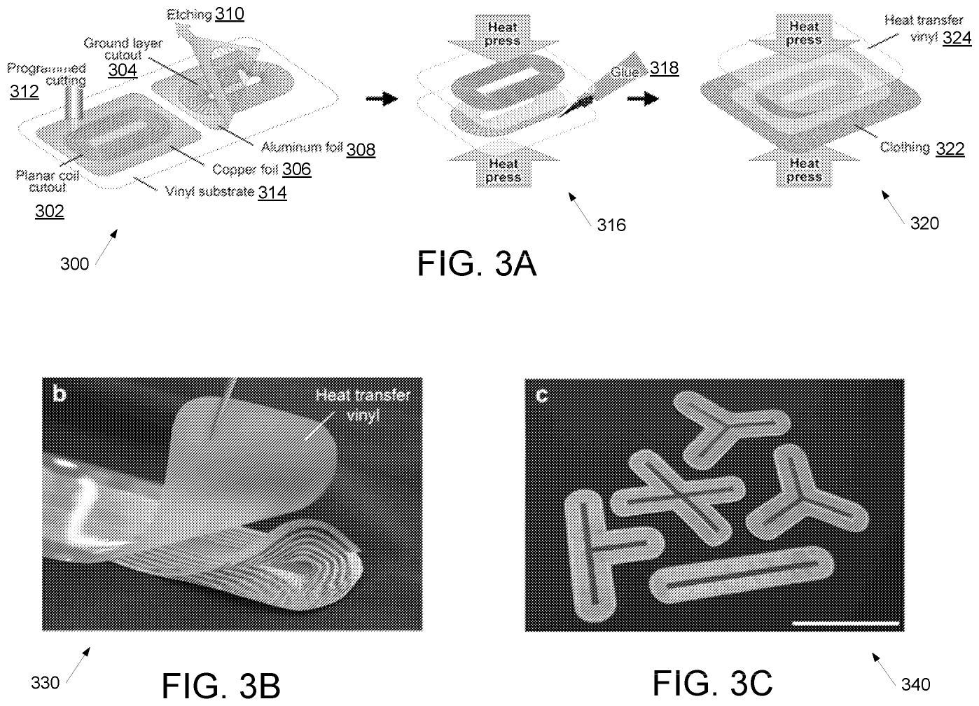

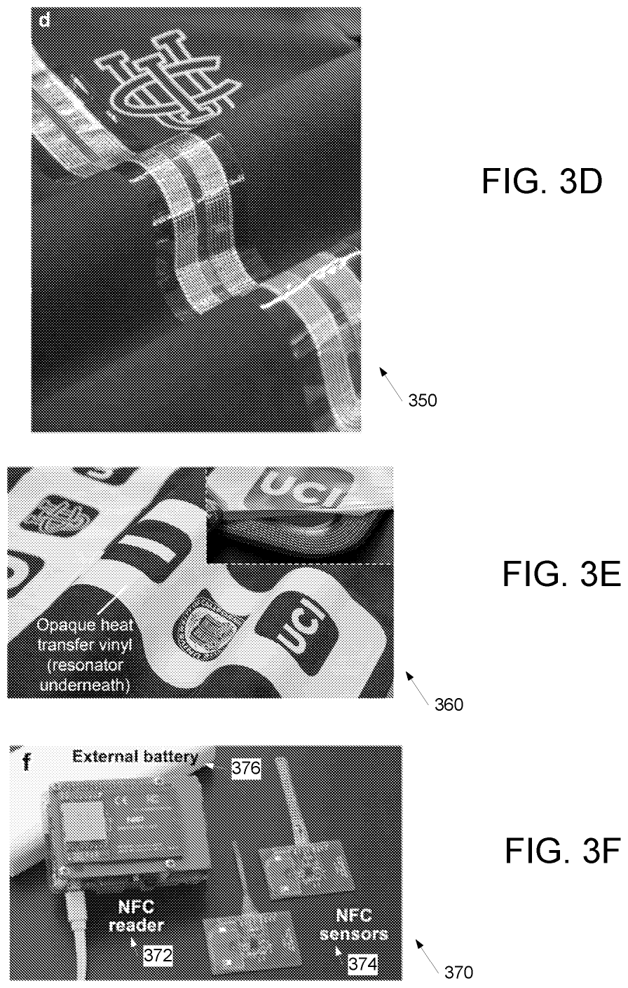

Schematic diagrams 300 illustrating resonator fabrication steps in accordance with an embodiment of the invention are shown in FIG. 3a. The coil trace 302 and slotted ground layer 304 may be cut out (e.g., programmed cutting 312) of copper 306 and aluminum foils 308 on vinyl substrate 314, respectively. The copper coils may minimize the network attenuation and ohmic loss per resonator as compared to aluminum (1.5Ω versus 3Ω for the configuration shown in FIG. 8), which in addition to increasing the resonator's quality factor, enhances the network's tolerance for misalignments and possible resonator mistuning. After etching 310, the layers may be first stacked (and heat pressed 316), then placed on the clothing 322, and finally fixed by heat pressing 320 (see also FIG. 3b-3d). In some embodiments, heat pressing may include the use of glue 318 and/or heat transfer vinyl 324. A diagram 320 illustrating a close up image of a resonator stack in accordance with an embodiment of the invention is shown in FIG. 3b. A diagram 340 illustrating various implementations of flexible resonators for optimal signal transmission and power division (scalebar 10 cm) in accordance with an embodiment of the invention FIG. 3c. A diagram 350 illustrating a wearable modular network integrated into clothing and covered by transparent heat transfer vinyl as a mechanical fixture in accordance with an embodiment of the invention is shown in FIG. 3d. This simple resonator fabrication allows for unique/specialized network designs. For example, wider coils may be utilized to increase the textile area covered by the nearfield propagation through the network. In addition to transparent vinyl, colored opaque vinyl can be used to conceal the BAN and integrate with special vinyl designs, thus embedding NFC transfer capabilities underneath customizable clothing designs (see FIG. 3e). A diagram 350 illustrating resonators that may be designed to cover wider nearfield areas and embedded underneath colored special vinyl designs in accordance with an embodiment of the invention is shown in FIG. 3e. This may allow network customizability both for function and style. The fabrication processes are further illustrated in Methods, as further described below.

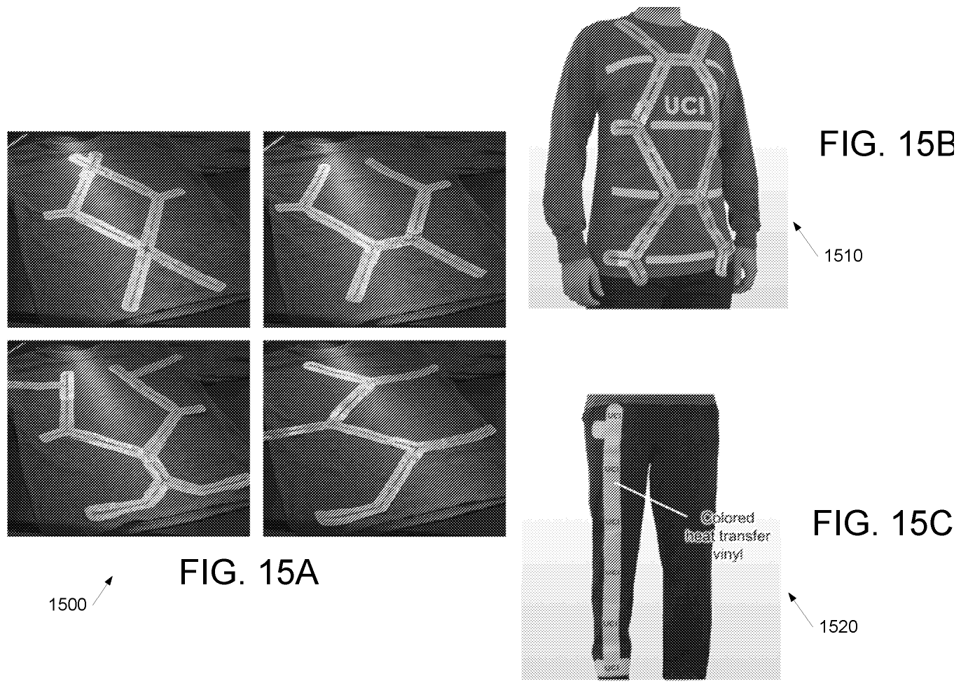

Numerous shirt- and pants-integrated network architectures with transparent and colored vinyl designs may be fabricated, which allow the network designs to integrate smoothly alongside traditional vinyl t-shirt designs. For example, various shirt networks 1500 enabled by complex parallel and closed loop metamaterial paths in accordance with an embodiment of the invention is shown in FIG. 15a. A colored vinyl implementation 1510 that may be utilized to appear next to the network in accordance with an embodiment of the invention is shown in FIG. 15b. A colored vinyl implementation 1520 that may be utilized to conceal the network underneath in accordance with an embodiment of the invention is shown in FIG. 15c.

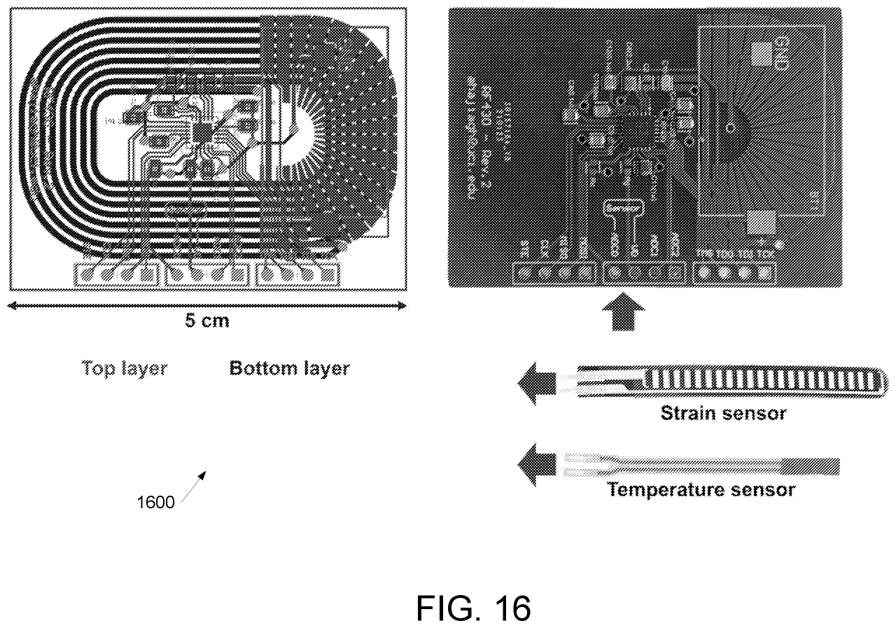

A diagram 370 illustrating battery-free NFC transponders integrated with strain and temperature sensors transferring respective sensor status to an NFC reader in accordance with an embodiment of the invention is shown in FIG. 3f. The versatility of the network was evaluated by various readers 372 and sensors 274 (including off-the-shelf chips and an optimized board design) shown in FIG. 3f, that can be placed close (roughly within the vertical distance of 3 cm) to any point of the resonator chain. In some embodiments, the sensor board may be based on a commercially available NFC transponder chip integrating an analog to digital unit that connects to a wide range of analog sensors, such as strain and temperature sensors. In some embodiments, the battery 376 may be optional. A diagram 1600 illustrating a board design of an NFC transponder in accordance with an embodiment of the invention is shown in FIG. 16. The loop antenna on the bottom layer may be designed to attain maximal coupling factor to the resonator's design. In many embodiments, the multiple NFC transponder access and readout may be implemented by both an NFC enabled mobile phone and programmed hardware as further described below.

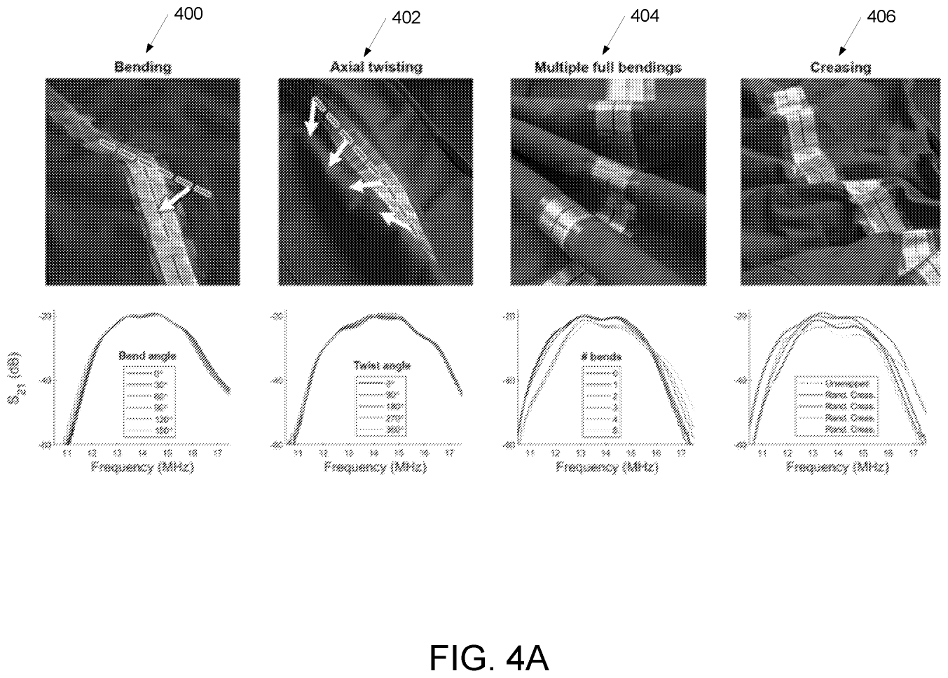

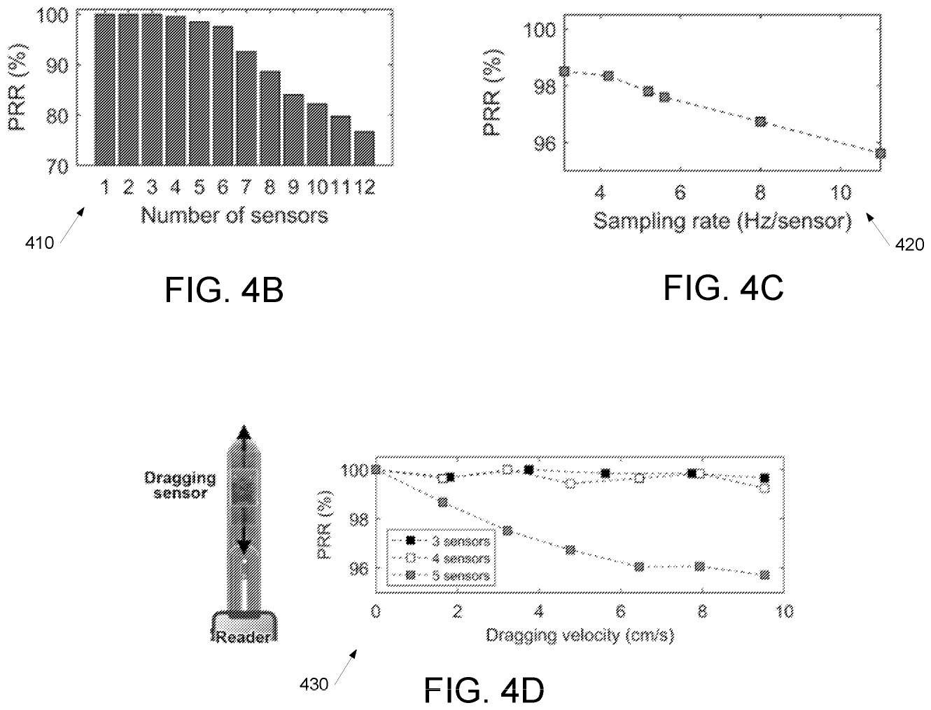

The flexibility of resonator elements exhibiting complex designs may be improved by employing thin aluminum, copper, and vinyl coatings. Each fabricated resonator element may have an overall thickness of about 300 μm and does not impede routine clothing movements. The spectral stability of a resonator array (80 cm long and integrated into cotton clothing) was examined by measuring the transmission (S₂₁) along the two ends of the network under various levels of bending and axial twisting. This shows a steady transmission with a minimum bandwidth of 16%. The functionality of the BAN may be tested under more severe mechanical deformations such as multiple full (360°) bendings and random creasings. The repeatable stability of the BAN was examined under mechanical distress by creasing at random locations along the network (followed by immediate unwrapping and repeating several times) and is shown in FIG. 4a. Specifically, a flexible, drag-and-drop NFC networks on textiles including spectral stability of clothing integrated pathways under mechanical deformation such as bending 400, axial twisting 402 (per meter length), multiple 360° bending 404, and repeated creasing/unwrapping 406 in accordance with an embodiment of the invention is shown in FIG. 4a. The 80 cm-long deformed inline network passes the transmission and bandwidth requirements enforced by NFC protocols.

The present embodiments may utilize software-based time domain multiple access (TDMA) to realize switching between sensing nodes. The controlled surface propagation of magneto-inductive waves eliminates the need for multiple nearfield antennas (connected by wire), or complex antenna switching schemes (needing active microelectronics). This allows conventional NFC-enabled smartphones to operate as compatible readers. The network's wireless efficiency was examined by measuring the NFC packet reception ratio (PRR), defined as the ratio of the number of packets successfully received by the reader to the total number of transmitted packets. Each packet may include sensor information from all transponders along the network during one refresh. We note that in practice, however, strain sensor latency and hysteresis may limit its performance at higher frequencies (see Methods section below). In addition, the TDMA approach allows up to 12 sensors to be connected along the network. There may be a tradeoff between the sampling rate, number of sensors, and packet loss. The PRR is measured for varying numbers of sensors in reach of the reader (with a sample rate of 5.6 Hz per sensor), as well as for various sampling rates (with 6 sensors) in an inline network of an overall length of 100 cm (see FIGS. 4b and 4c). A diagram 410 illustrating averaged NFC PRR measured for various numbers of sensor-transponders along a network with a refresh rate of 5.6 Hz per sensor (and distributed over 1 m of a network) in accordance with an embodiment of the invention is shown in FIG. 4b. A diagram 420 illustrating PRR for 6 NFC transponders with various sampling rates in accordance with an embodiment of the invention is shown in FIG. 4c.

The ability to continuously monitor wireless NFC-enabled devices all-along the magneto-inductive resonator railway offers useful applications in unique scenarios such as, but not limited to, moving joints within actuators/robotics (which cannot be powered through NFC's regular range), or within highly traceable terminals within gates wherein multiple objects may locomote along a local pathlength (where traditional radiofrequency identification would again fail). To validate the drag-and-drop feature of the BAN, we power and probe sensors moving along the network (1 m length) at different velocities and various numbers of sensors within reach. The surface-bound magnetic profile of the planar magneto-inductive array allows for relatively fast sensor dragging with a reliable PRR of above 95% (see FIG. 4d). A diagram 430 illustrating packet loss for a moving sensor dragged under various velocities along 1 m of the magneto-inductive network for different numbers of sensors under a sampling rate of 9 Hz/sensor in accordance with an embodiment of the invention is shown in FIG. 4d.

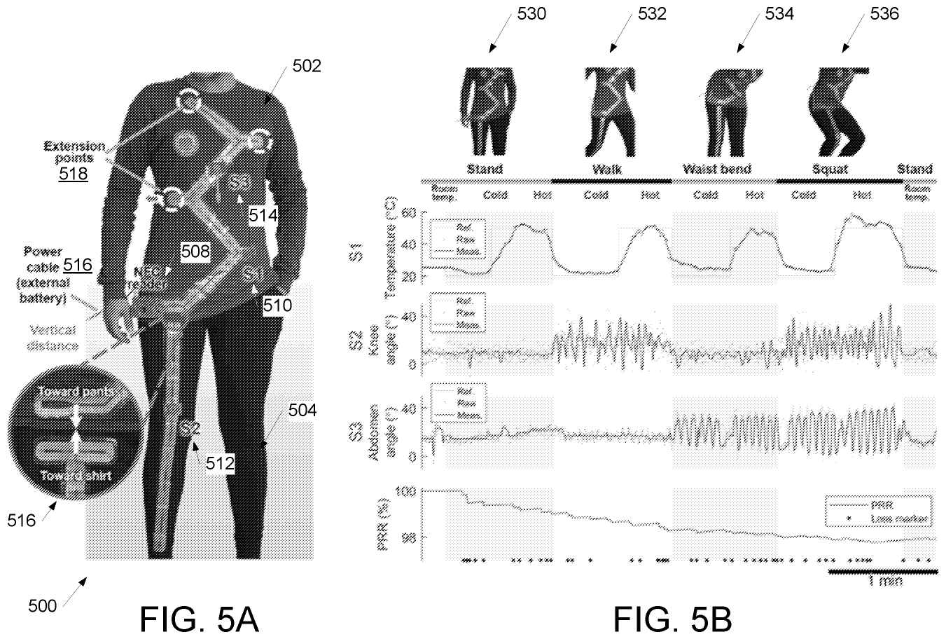

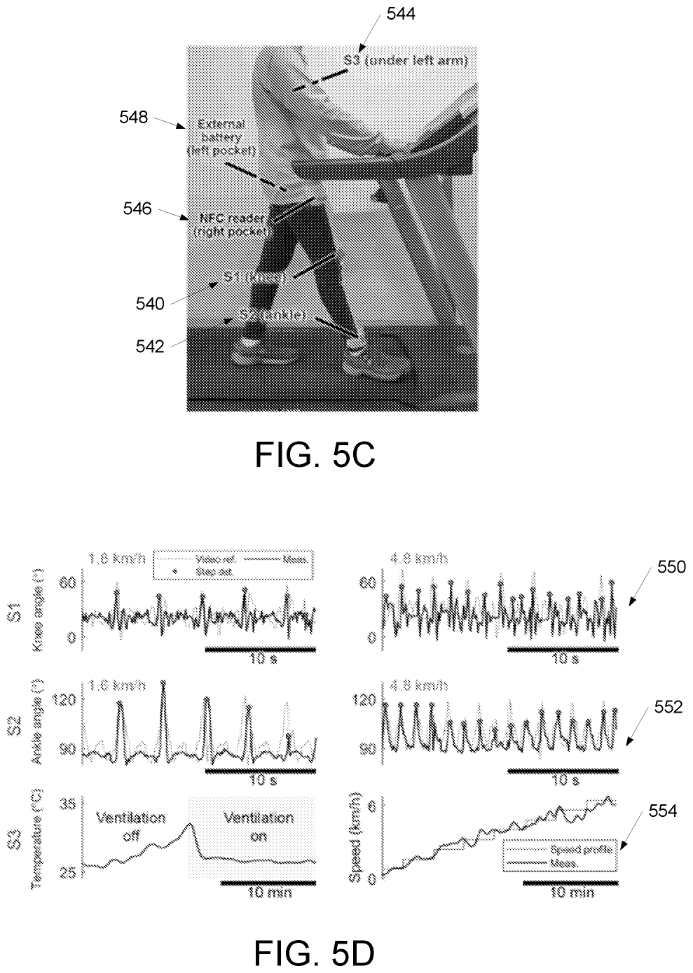

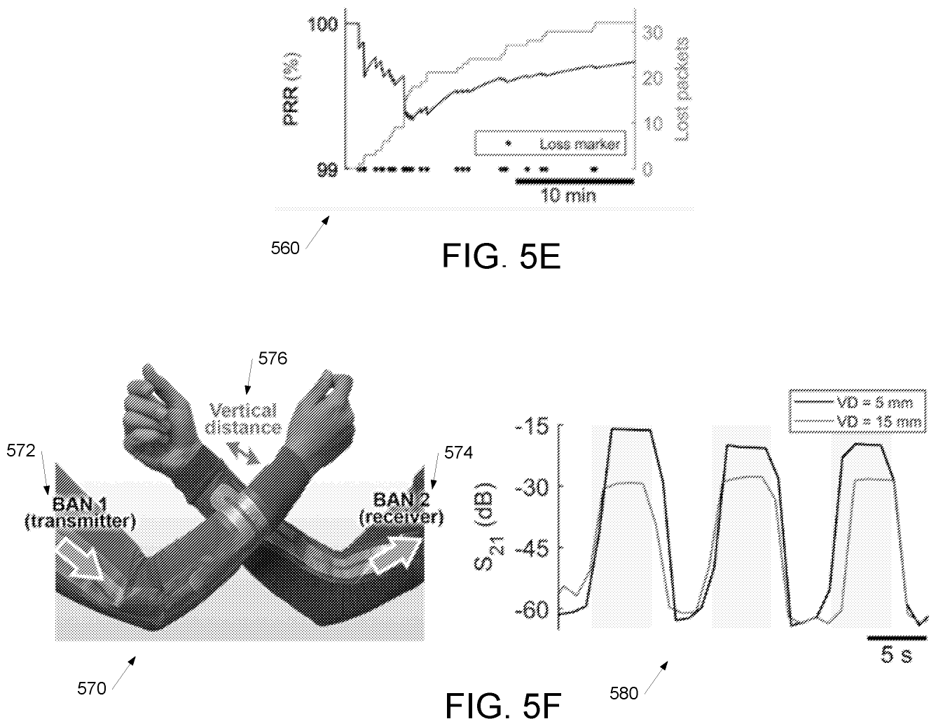

In many embodiments, the upper body part of the present BANs may be implemented in a zigzag shape, passing over the abdomen and chest area, while offering the ability to extend the network to the back of the body (potentially interacting with NFC-enabled seats). Similarly, an inline array may be implemented starting from the hip, passing over the knee, and ending by the ankle. The shirt's network ending may be implanted to overlap with that of the pants within a vertical distance, thus enabling wireless signaling across different pieces of clothing (see FIG. 5a). A diagram 500 illustrating a body area network (BAN) utilizing multi-transponder and multi-BAN communication by textile-integrated waveguides on a shirt 502 and pants 504 and the pants/shirt terminals 506 in accordance with an embodiment of the invention is shown in FIG. 5a. In many embodiments, the NFC reader 508 may receive information from multiple sensors 510, 512, 514, and may be connected to an external battery 516. In many embodiments, the BAN may include one or more extensions points 518. For detailed sensor placements see the Methods section below.

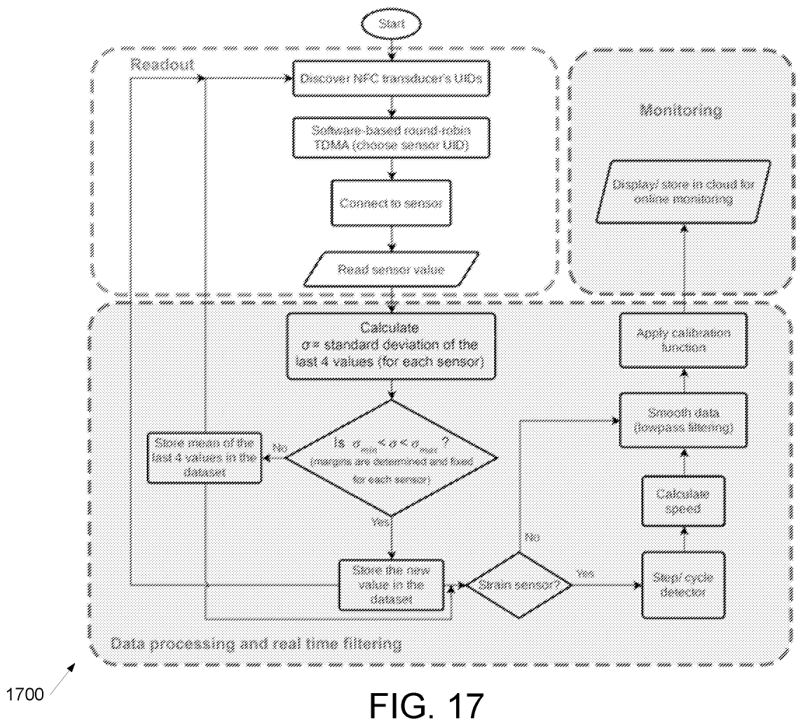

In various embodiments, the multipoint sensor readout may be performed while standing, walking, squatting, and waist-bending at different temperature zones simulated indoors. All sensor values may be timestamped, transmitted, and recorded by the reader at various rates including, but not limited to, a sampling rate of 5.6 Hz/sensor (equivalent to an overall rate of 17 Hz for all sensors operating together). To minimize the resistive strain and temperature sensor's hysteresis effect and random noises, a data processing algorithm may be applied for each sensor value (processing flowchart is shown in FIG. 17). A flowchart 1700 illustrating software based TDMA and sensor data processing in accordance with an embodiment of the invention is shown in FIG. 17. The sensor value storage decision (based on the standard deviation) allows for holding the sensor values close to the previously logged data, ultimately rejecting artifacts caused by the analog sensor noise in real time and without inducing considerable latency. The SD margin and calibration function is unique to each sensor type and does not change throughout the monitoring process.

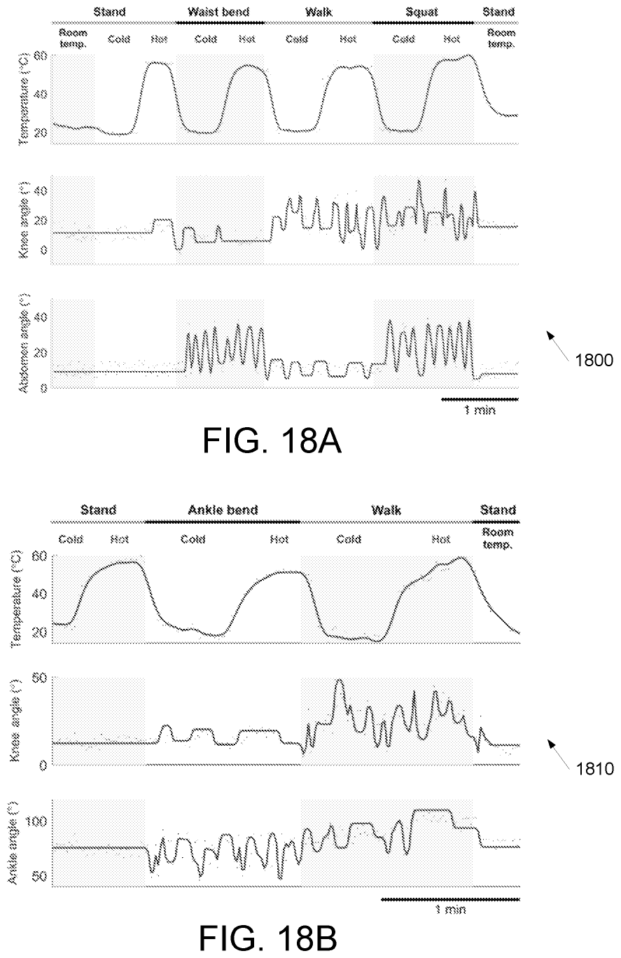

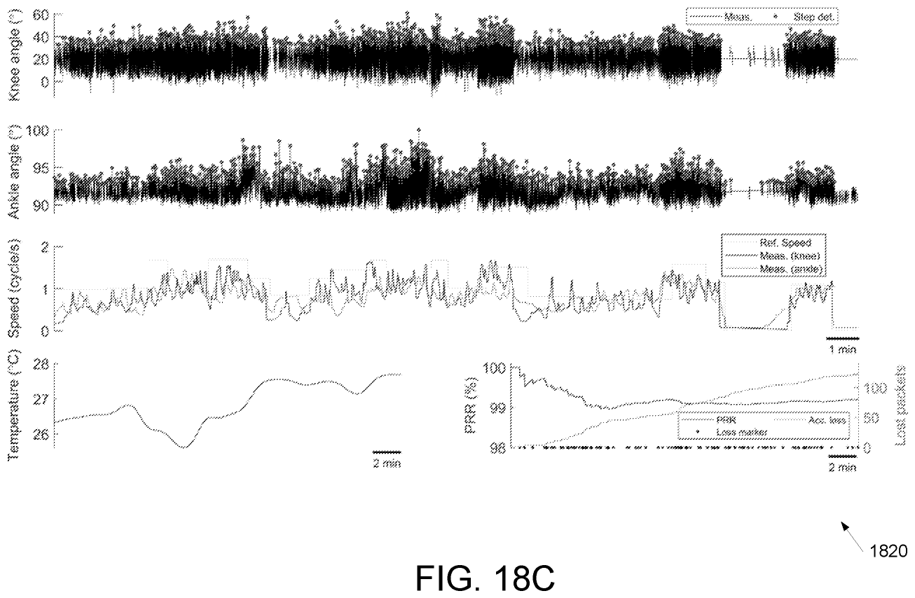

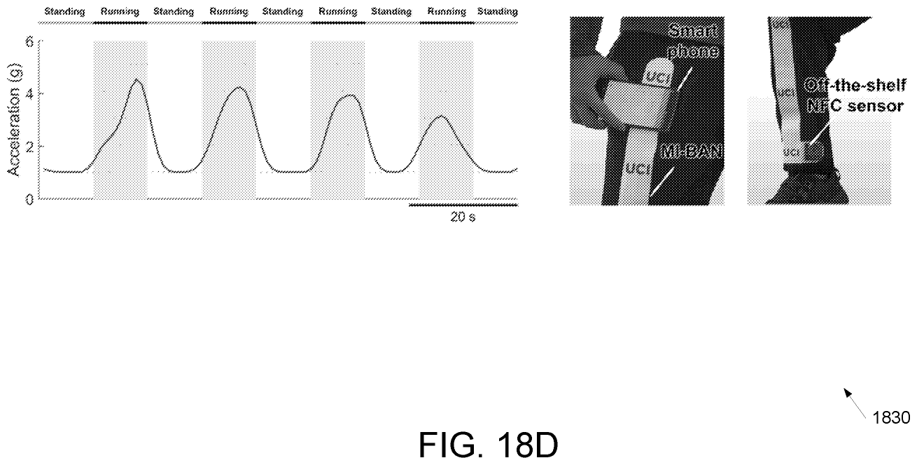

The above strategy for minimizing the resistive strain and temperature sensor's hysteresis effect and random noises may be implemented in real time and a few seconds after the start of recording (see methods section for filtering details). This approach effectively achieved steady data with minimal noise fluctuations. A real time short-term and low-speed monitoring of human activity realized by time-division based multiple sensor readout within the BAN with a sampling rate of 5.6 Hz/sensor (dots and solid lines show raw and filtered data, respective) in accordance with an embodiment of the invention is shown in FIG. 5b. The sensor's raw and filtered samples during standing 530 a short-term exercise (walking for 8 steps 532, 6 bends 534, and 7 squats 536, each in two different temperature zone) are illustrated. The cold/hot temperature zones correspond to locally different but close indoor spots generated by a fan/heater (the temperature profile sampled by an infrared thermometer). For applications with conservative power consumption, low sampling rate versions of the exercises are performed as well (FIGS. 18a and 18b). The network's functionality may be examined with different sensor placements, an NFC enabled mobile phone, and off-the-shelf sensor/transponders. FIG. 18 illustrates additional in vivo tests of the network's versatility at various sampling rates and for off-the-shelf NFC sensor/smartphone. Diagrams 1800 illustrating full body (temperature sensor on the belly, and strain sensors on knee and ankle) movement detection at a low sampling rate (1 Hz/sensor or 3 Hz overall) in accordance with an embodiment of the invention is shown in FIG. 18a. Diagrams 1810 illustrating lower body (temperature sensor on the hip, and strain sensors on knee and abdomen) movement detection at a low sampling rate (1 Hz/sensor or 3 Hz overall) in accordance with an embodiment of the invention is shown in FIG. 18b. Diagrams 1820 illustrating lower body cycling detection at a higher sampling rate (5.6 Hz/sensor) in accordance with an embodiment of the invention is shown in FIG. 18c. Diagrams 1830 illustrating test of network's compatibility with off-the-shelf sensor and compatible application (e.g., Android application) (dots and solid lines show raw and filtered data, respectively) in accordance with an embodiment of the invention is shown in FIG. 18d.

A high speed, long-term indoor walk/running activity measurement in accordance with an embodiment of the invention is shown in FIG. 5c. In many embodiments, test may be performed under a gradually increasing velocity profile for 25 min and BAN may be integrated into clothing with colored vinyl. In various embodiments, the BAN may include sensors 540, 542, 544, and NFC reader 546. In some embodiments, the BAN may also include an external battery 548. Diagrams 550, 552, 554, monitoring of sensors 540, 542, 544, respectively, during indoor running under various velocity profiles with a sampling rate of 10 Hz/sensor in accordance with an embodiment of the invention is shown in FIG. 5d. In various embodiments, steps may be detected and marked by circular markers. Diagram 560 illustrating long-term packet loss monitoring during indoor running in accordance with an embodiment of the invention is shown in FIG. 5e.

Interestingly, the surface propagation characteristics of the magneto-inductive structures enable seamless body-to-body communication with no need for terminals. The link readily establishes by putting any point of the two contributing BANs close enough (similar to the vertical distance between different pieces of clothing as shown in FIG. 5f). A diagram 570 illustrating body-to-body communication enabled by NFC's plug-and-play characteristics and its measured transmission during dragging the hands close and far over time (comprising an action of a “digital high-five”) for various VD values in accordance with an embodiment of the invention is shown in FIG. 5f. The body-to-body communication may include a first BAN (transmitter) 572 and a second BAN (receiver) 574 having a vertical distance 576. The external near-field communication may similarly be generalized to nearby local networks integrated into, for example, driver seats or gateways for monitoring and authentication purposes. The graph 580 illustrates data for VD=5 mm and 15 mm.

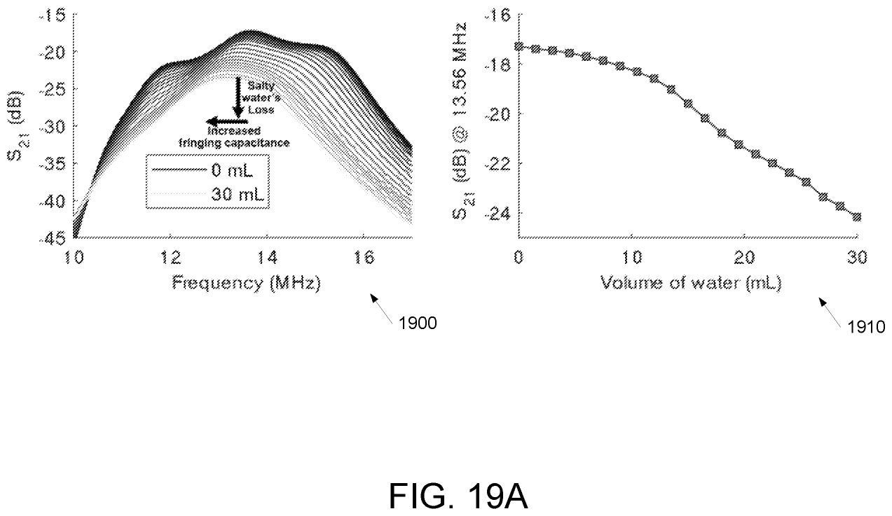

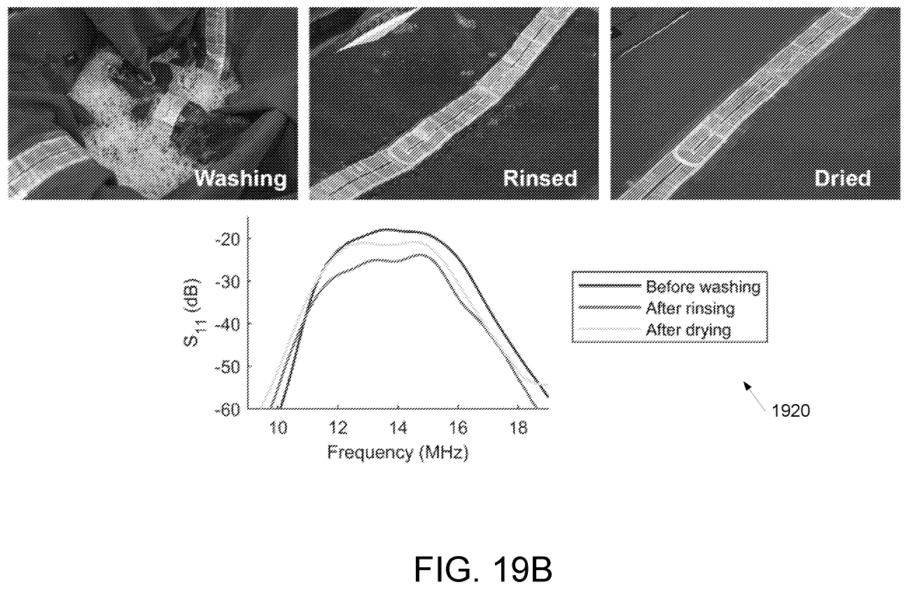

Due to the vinyl sealing of the network, it demonstrates a good spectral stability versus wetting. To mimic human sweating (or light raindrops), we added incremental volumes of salty water (30 mL total, with NaCl concentration of 200 mg/L) on top of the network (covering about 90 cm² of the resonators), resulting in 3% frequency shift and maintaining steady network transmission. Additionally, the textile survived 20 minutes of handwashing in cold water with stable measured transmission over various washing phases (see FIG. 19). FIG. 19 illustrates spectral stability versus wetting and washing. Diagrams 1900, 1910 illustrating frequency and magnitude shifts caused by high permittivity (increasing the fringing capacitance of the resonators) and loss of the added water in accordance with an embodiment of the invention is shown in FIG. 19a. Incremental doses (150 μL) of a stock NaCl solution (200 mg/L) were added to mimic the electrical conductivity of human sweat or raindrops. Diagrams 1920 illustrating robustness of the magneto-inductive BAN's spectral characteristics during different phases of washing is shown in FIG. 19b. The handwashing test was performed with cold water (room temperature) and a total time of 20 minutes.

The magneto-inductive BAN may be synthesized on-demand and at a low cost for personalized wearable networks. This ecosystem may be utilized in various clinical, athletic, and daily routines to facilitate real-time healthcare and status monitoring. For instance, integration into hospital patient uniforms may allow for seamless patient monitoring where sensors are dragged-and-dropped across clothing. Professional sports clubs or federations may develop highly customized networks that may be both integrated with their branding and optimized to serve specific needs in athletic training and monitoring. The vinyl-based elements enable users to freely create and readily arrange the network without special equipment, and may be targeted for either local or long-range monitoring along the body. Sensing nodes may be swapped or rotated seamlessly to facilitate plug-and-play measurement of a variety of relevant parameters. Lastly, the magneto-inductive BAN is compared with some of recent over-the-air network technologies (see Table S1, reproduced below).

Although specific clothing integration and multi-sensor studies are discussed above with respect to FIGS. 3a-5f and 12-19b, any of a variety of clothing integration and multi-sensor studies as appropriate to the requirements of a specific application can be utilized in accordance with embodiments of the invention. Methods and design considerations in accordance with embodiments of the invention are discussed further below.

TABLE S1
Comparison of recent battery-free over-the-air BAN technologies.

| | Magneto- | | | | |
|---|---|---|---|---|---|
| | inductive | | | | |
| | metamaterials | Spoof surface | | | |
| | (present | plasmon | Coils connected | | |
| Technology | embodiments) | metamaterials | by wire | Qi powered | RFID |
| Standard | NFC | Bluetooth | NFC | Qi | Specialized |
| Frequency | 13.56 MHz | 2.5 GHz | 13.56 MHz | 142 kHz | >10 MHz |
| Propagation | Bound to surface | Bound to surface | Bound to surface | Bound to | Prone to third |
| security | | (for full radiative | | surface | party listening |
| | | to guided mode | | | |
| | | redirection) | | | |
| Operational | Radius of ~1 m | Radius of ~1 m | Equal to wire | Equal to wire | Up to 25 mm at |
| range | (drag-and-drop | (drag-and-drop | length (drag-and- | length (drag-and- | 13.56 MHz |
| | available) | available) | drop unavailable) | drop unavailable) | (lower range for |
| | | | | | higher frequencies) |
| Clothing | Easy (inexpensive | Easy (relatively | Difficult | Easy (cheap | Not applicable |
| integration | vinyl resonators | expensive | (embroidered rails | wires attached | |
| difficulty (for | heat transferred | conductive fabric | not available | on clothing) | |
| the end-user) | on pre-existing | and adhesive) | on pre-existing | | |
| | clothing) | | clothing) | | |
| Sensitivity to | Insensitive (low | Sensitive | Sensitive | Sensitive | Sensitive |
| body proximity | loss integrated | (relatively higher | (potentially | | |
| | ground layer) | at microwave band) | reducible by | | |
| | | | appropriate | | |
| | | | grounding) | | |
| Multi-clothing | Up to 30 mm, no | Up to 10 mm, no | Up to ~30 mm, | Up to ~10 mm, | Not applicable |
| (vertical distance) | need for terminals | need for terminals | needs terminals | needs terminals | |
| communication | (low loss | (relatively higher | | | |
| | associated with | loss associated with | | | |
| | discontinuities) | discontinuities) | | | |
| On-demand | Available (no | Unavailable | Unavailable | Unavailable | Unavailable |
| extension | need for | (considerable loss | (conductive | | |
| | electrical/ | upon discontinuity | thread sewing | | |
| | mechanical | made by attachments) | needed) | | |
| | connection) | | | | |
| Reader/sensor | Anywhere along | Anywhere along | Only on the BAN's | Only on the BAN's | Only on the |
| (or multi-BAN) | the BAN | the BAN | hub/terminals | hub/terminals | sensor |
| placement | | | | | |
| Versatility | Compatible with | Compatible with | Compatible with | Compatible with | Specialized |
| | standard | standard | standard | standard | reader/sensor |
| | | | | | communication |

### Methods and Design Considerations

Numerical methods. The equivalent circuit's behavior may be modeled in MATLAB and validated in Advanced Design System, Keysight. The FEM simulations of the grounded resonators (including the magnetic field profile demonstration) may be conducted in COMSOL Multiphysics (using electromagnetic waves, and magnetic field physics).

Resonator fabrication. The grounded resonators may be fabricated by stacking a copper sheet (20 μm thick) on a transparent self-adhesive vinyl film substrate. Similarly, aluminum foil (14 μm thick) may be used for the ground layer. The metal/vinyl layers may be stacked on the adhesive cutting mat and cut using Silhouette Cameo 3 (Silhouette America Inc., Lindon, Utah, USA). The complement pattern of the metal foil may be etched after cutting. Then the top aluminum surface of the ground stack may be coated with general purpose adhesive spray. The coil (copper/vinyl stack) may be then aligned and placed on top of the coated surface of the ground layer (aluminum/vinyl stack) after one minute. The final stack (copper/vinyl/aluminum/vinyl) may then immediately covered by inflammable clothing and heat pressed under 260° F. for 45 seconds and let cool afterward. The adhesive coating should be uniform and controlled to avoid resonance frequency shifts. The resonance behavior of the grounded coils may then be measured (and tuned if necessary) with a VNA (E5063A, Keysight) linked to a loop antenna via an SMA connector to ensure all elements resonate at 13.56 MHz to achieve the maximum transmission (a frequency mistuning of about 0.3 MHz was found to be tolerable). The tuning may be performed by etching the copper trace (lower loop length results in lower L_{R} and C_{R} which increases the resonance). Optionally, an additional transparent vinyl may be placed on top of the resonator (adhesive facing copper) to seal the resonator and enhance mechanical and waterproof properties. The ground layer should be of the same shape as the resonating loop and cover its entire area. We added a 1 mm margin to suppress the fringing electric fields (from the resonator to the body) as well. The ground layer's gap distances should be as small as a millimeter to eliminate the eddy currents efficiently (see FIG. 6).

Systematic design procedure of magneto-inductive networks. After designing the resonator such that it resonates at 13.56 MHz, we measured the electrical characteristics (impedance) of the identical resonators and substituted them in our model to find the approximate k_{RR} value based on the dispersion diagrams. For our rectangular coil geometry, the k_{RR}=0.1 maps onto 3.2 cm of neighbor loop overlap (or d_{c}=12 cm). One may redesign the loop resonator to match a particular clothing shape or application, resulting in a different k_{RR}, and thus d_{c}.

Textile integration. After locating and placing the resonators on top of the clothing, a heat transfer vinyl (transparent or opaque depending on the design preference) may be cut and put on top, and then heat pressed under 300° F. for 1 min and let cool afterward. Cotton clothing is often suggested by vinyl manufacturers due to its tolerance to high temperatures.

NFC sensor transponder. The sensor ecosystem may be implemented using ISO15693 sensor transponder RF430FRL152H, Texas Instruments (TI) with unique identifications (UID), and designed using a miniature board containing NFC loop antennas (to couple better with the resonators) and analog sensor biasing resistors to minimize the analog to digital (ADC) gain error. The chips may then be programmed over-the-air to announce the ADC output (known as the sensor value) upon the reader's interrogation command under ISO15693. The programming may be performed using TI's GUI to interface between the TRF7970A, TI (mounted on MSP430G2553, TI), and the transponder chip. An off-the-shelf strain (Short Flex Sensor, Adafruit Industries LLC) and temperature (CTTS-203856-S02, Amphenol Advanced Sensors) sensors may be used and embedded in the transponders. The sampling rate (32 Hz) here may be significantly higher in comparison to prior NFC multiplexing techniques (8 Hz) for a single sensor. The strain sensor (here used under 30% strain, enough to measure knee/ankle bending during activities) showed less than 200 ms latency, which allowed a sampling rate of 5.6 Hz/sensor. The sampling rates for activity measurement (5.6 Hz/sensor) were set based on the PRR (above 95% for at least 5 sensors) and the Nyquist sampling theorem (that the sampling rate must be over twice as fast as the activity's highest frequency component, which was estimated to be less than 2.5 cycles/steps per second). The PRR studies (shown in FIGS. 4b and 4c) were run for 10 min (for each number of sensor and each sampling rate) to ensure the PRR values reflect the steady-state of the network.

Multi-sensor readout and reliability check. To evaluate the worst-case transmission scenario, any packet with at least one failed sensor data (even if all other sensor data is delivered successfully) is counted as lost. Our software-based TDMA (integrated into an NFC pathway ecosystem with sensing nodes 1 m away from the central reader) reached a maximum refresh rate of 32 Hz for one sensor while exhibiting a PRR of above 98%. This approach allows for a wide array of sensor types for frequent monitoring (to maintain the same PRR with a larger number of sensors, the sampling rate should be lowered accordingly). NXP PN7150 NFC controller shield mounted on Raspberry Pi may be utilized to perform the sensor readout, which broadcasts the reading command along the BAN. To avoid collision among the transponders receiving this command, the multiple access may be implemented with round-robin scheduling to enable time division between the NFC transponders discovered at the time and looped until receiving the termination command. For sensors with higher readout priority or sampling rate, the rate-monotonic scheduling might be beneficial.

Multi clothing design and sensor placements. Since the pants network passes over the pocket, the NFC reader could be placed in the pocket for appropriate excitation, similar to mobile devices routinely placed in pockets. For basic human daily activity pattern measurement, we placed two strain sensors at the abdomen area and knee (for bending and pacing recognition) and a temperature sensor above the hip (recording near-body temperature), with sensing nodes located in the vicinity of the network. The standalone reader (placed on the pants pocket) was powered up via a wired external battery, and sensor data was streamed wirelessly to the online cloud. Here, the co-location of sensors may not matter as long as they are within the reader's sensitivity range (equivalent to the transmission above approximately-35 dB here).

Long term activity validation. The indoor walk/run was performed under varying speed profiles and a locally-controlled ventilation system nearby and sensors were probed at a sampling rate of 5.6 Hz/sensor. The knee and ankle angles obtained from the BAN-based measurement were compared with a reference video processing toolbox (video motion-capture) to validate measured actions. The running test was repeated three times (each 25 min) under room temperature, in which the ventilation was turned on in the middle of the experiment. Here, the core body temperature correlate increased during more strenuous activity, and expectedly reduced (to room temperature) when ventilation was initiated.

Off-the-shelf NFC transponders. The BAN's versatility test may be performed using STEVAL-SMARTAG1 NFC transponders. Among the embedded pressure, temperature, humidity, and accelerometer sensors, the latter may be utilized to detect running while placing the transponder on the ankle. The readout may be performed with ST NFC Sensor application on a mobile device (e.g., an Android device).

Verification of activity monitoring. The video tracking of human activity tests subject may be performed using OpenCV library implemented by Python programming. The measured temperatures may be verified with an infrared thermometer. The in-vivo exercise tests were run by a male human (172 cm tall and 62 kg).

Real time sensor data processing. The real time filtering operates based on the standard deviation (SD) of the last 4 samples (independently for each sensor) and decides to filter or pass the incoming data depending on the fluctuations. The minimum and maximum bounds of the acceptable SD are found by the strength of the random noise and extreme fluctuations (such as dropped packet or analog sensor overflow), respectively. Any sensor values between these boundaries are passed, or otherwise the mean value of the last 4 samples (including the new sample) is stored to hold the acceptable data. The thresholds vary based on the sensor's noise characteristics and obtained by calibration, and were not altered through the experiments.

Although specific methods and design considerations are discussed above, any of a variety of methods and designs as appropriate to the requirements of a specific application can be utilized in accordance with embodiments of the invention. While the above description contains many specific embodiments of the invention, these should not be construed as limitations on the scope of the invention, but rather as an example of one embodiment thereof. It is therefore to be understood that the present invention may be practiced otherwise than specifically described, without departing from the scope and spirit of the present invention. Thus, embodiments of the present invention should be considered in all respects as illustrative and not restrictive.

What is claimed is:

1. A body area network, comprising:
a first array of magnetically coupled resonators configured to propagate magneto-inductive (MI) surface waves;
wherein the first array of magnetically coupled resonators comprises a plurality of MI elements; and
wherein the first array of magnetically coupled resonators creates a flexible magnetic metamaterial path for wireless communication using the MI surface waves.

2. The body area network of claim 1, wherein the plurality of MI elements is tuned to near field communication (NFC) bandwidths.

3. The body area network of claim 2, wherein the first array of magnetically coupled resonators is configured to generate NFC-based MI surface waves.

4. The body area network of claim 1, wherein the body area network utilizes pairing and security of NFC protocol.

5. The body area network of claim 1 further comprising at least one NFC enabled sensor connected to the first array of magnetically coupled resonators.

6. The body area network of claim 5, wherein a time-division, multiple access protocol is implemented to interrogate the at least one NFC enabled sensor.

7. The body area network of claim 6 further comprising an NFC enabled reader.

8. The body area network of claim 7, wherein the NFC enabled reader is a mobile device.

9. The body area network of claim 1, wherein each of the plurality of MI elements comprises a flexible planar coil.

10. The body area network of claim 9, wherein the flexible planar coil is cut out of copper.

11. The body area network of claim 9, wherein each of the plurality of MI elements comprises a ground layer that minimizes spectral uncertainly due to a human body's parasitic effect.

12. The body area network of claim 11, wherein the ground layer includes at least one slot perpendicular to the flexible planar coil.

13. The body area network of claim 11, wherein the ground layer is cut out of aluminum foil.

14. The body area network of claim 11, wherein the first array of magnetically coupled resonators is integrated into a first clothing textile by stacking the flexible planar coil on the ground layer and adhering the ground layer to the first clothing textile using a heat press process.

15. The body area network of claim 14, wherein the heat press process includes applying heat to at least one heat transfer vinyl.

16. The body area network of claim 1 further comprising a second array of magnetically coupled resonators, wherein the second array of magnetically coupled resonators comprises a plurality of MI elements.

17. The body area network of claim 16, wherein the first array of magnetically coupled resonators is integrated into a first clothing textile and the second array of magnetically couple resonators is integrated into a second clothing textile.

18. The body area network of claim 17, wherein the first array of magnetically coupled resonators and the second array of magnetically coupled resonators are separated by a clothing transition between the first clothing textile and the second clothing textile.

19. The body area network of claim 18, wherein the MI surface waves generate a wireless communication link across the clothing transition.

20. The body area network of claim 1, wherein the MI surface waves generate a wireless communication link between the first array of magnetically coupled resonators and at least one object.

21. The body area network of claim 1, wherein the body area network is associated with a first person and the MI surface waves generate a wireless communication link between the body area network and an external array of magnetically coupled resonators associated with a second person.

22. The body area network of claim 1, wherein the plurality of MI elements is connected in series.

23. The body area network of claim 1, wherein the plurality of MI elements is connected in parallel.
